(19) United States
(12) Patent Application Publication
(10) Pub. No.: US 2022/0227850 A1
(43) Pub. Date: Jul. 21, 2022

(54) BINDING MOLECULES

(71) Applicant: CRESCENDO BIOLOGICS LIMITED, Cambridge (GB)

(72) Inventors: Grainne DUNLEVY, Cambridge (GB); Colette JOHNSTON, Cambridge (GB); Daniela SYDORUK, Cambridge (GB); Martyna LEWANDOWSKA, Cambridge (GB)

(21) Appl. No.: 17/610,328

(22) PCT Filed: May 15, 2020
(86) PCT No.: PCT/GB2020/051199

§ 371 Date: Nov. 10, 2021

(30) Foreign Application Priority Data
May 15, 2019 (GB) 1906870.9
May 15, 2019 (GB) 1906872.5

(51) Int. Cl. C07K 16/18

(52) U.S. Cl. CPC C07K 16/18; C07K 2317/569; A61K 2039/505; C07K 2317/94; C07K 2317/92; C07K 2319/31

(57) ABSTRACT

The disclosure relates to single domain antibodies that bind human serum albumin and methods for using such single domain antibodies to extend the half life of therapeutic molecules.

### INTRODUCTION

The pharmacokinetics of proteins and peptides is governed by the parameters of absorption, biodistribution, metabolism, and elimination. The most common routes of clearance for proteins and peptides include endocytosis and membrane transport-mediated clearance by liver hepatocytes for larger proteins, and glomerular filtration by the kidney for smaller proteins and peptides.

Many drugs that possess activities that could be useful for therapeutic and/or diagnostic purposes have limited value because they are rapidly eliminated from the body when administered. For example, many polypeptides that have therapeutically useful activities are rapidly cleared from the circulation via the kidney.

Accordingly, a large dose must be administered in order to achieve a desired therapeutic effect. A need exists for improved therapeutic and diagnostic agents that have improved pharmacokinetic properties.

Thus, different strategies have been employed to improve the pharmacokinetics of smaller proteins and peptides, including increasing the size and hydrodynamic radius of the protein or peptide, increasing the negative charge of the target protein or peptide or increasing the level of serum protein binding of the peptide or protein through binding to albumin. This includes fusion of the biologically active protein or peptide to human serum albumin (HSA), fusion to the constant fragment (Fc) domain of a human immunoglobulin (Ig) G or fusion to non-structured polypeptides such as XTEN (Reviewed in Stroh “Fusion Proteins for Half-Life Extension of Biologics as a Strategy to Make Biobetters BioDrugs”. 2015; 29(4): 215-239).

Different applications require different half life and there still exists a need to provide bespoke half life extending molecules. The invention is aimed at addressing this need.

### SUMMARY

The invention relates to immunoglobulin single variable domain antibodies that bind HSA, in particular human immunoglobulin single variable heavy chain domain antibodies, e.g. in particular human immunoglobulin single variable heavy chain domain antibodies obtained or obtainable from transgenic mice expressing unrearranged human V, D, J gene segments.

In one aspect, the invention relates to an immunoglobulin single variable domain antibody that binds HSA comprising or consisting of SEQ ID NO. 1 or a sequence with at least 80%, 90% or 95% homology thereto, SEQ ID NO. 30 or a sequence with at least 80%, 90% or 95% homology thereto or SEQ ID NO. 5 or a sequence with at least 80%, 90% or 95% homology thereto. The invention also relates to an immunoglobulin single variable domain that binds HSA which is a variant of SEQ ID NO. 1 and has 1 to 20 amino acid substitutions compared to SEQ ID NO. 1. The invention also relates to an immunoglobulin single variable domain that binds HSA which is a variant of SEQ ID NO. 5 and has 1 to 20 amino acid substitutions compared to SEQ ID NO. 5. In another aspect, the invention also relates to a method for extending the half life of a protein comprising joining said protein to an immunoglobulin single variable domain as described herein. The invention also relates to the use of an immunoglobulin single variable domain as described herein extending the half life of a therapeutic moiety when said immunoglobulin single variable domain antibody is linked to said therapeutic moiety in a fusion protein.

In another aspect, the invention relates to a pharmaceutical composition comprising an immunoglobulin single variable domain antibody as described herein or a protein or construct as described herein.

The invention also relates to a nucleic acid sequence that encodes an amino acid sequence as described herein.

The invention further relates to a vector comprising a nucleic acid sequence as described herein.

The invention also relates to a host cell comprising the nucleic acid sequence as described herein or a vector as described herein.

The invention also relates to a kit comprising an immunoglobulin single variable domain antibody as described herein or a protein or construct as described herein or a pharmaceutical composition as described herein.

Furthermore, the invention relates to a method for producing a heavy chain only antibody or a binding molecule comprising at least one human immunoglobulin single domain antibody capable of binding human HSA as described herein wherein said domain is a human V_{H} domain said method comprising

a) immunising a transgenic mouse with an HSA antigen wherein said mouse expresses a nucleic acid construct comprising human heavy chain V genes and is not capable of making functional endogenous light or heavy chains, b) generating a library of sequences comprising V_{H} domain sequences from said mouse and

c) isolating sequences comprising V_{H} domain sequences from said libraries.

### FIGURES

The invention is further illustrated in the following non-limiting figures.

FIG. 1. Serum levels after a single i.v. administration of TPP-1246 at 3 mg/kg in cynomolgus macaque.

### DETAILED DESCRIPTION

The embodiments of the invention will now be further described. In the following passages, different embodiments are described. Each aspect so defined may be combined with any other aspect or aspects unless clearly indicated to the contrary.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols 1 and 2, Ontermann and Dubel, eds., Springer-Verlag, Heidelberg (2010).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The present invention relates to amino acid sequences binding to human serum albumin (HSA) and binding molecules, such as proteins, that comprise such amino acid sequences. In particular, the invention relates to single domain antibodies or immunoglobulin single variable domains having the amino acids as described herein and which can be exploited in therapeutic methods and uses as well as in pharmaceutical formulations as described herein.

Single domain antibodies described herein bind specifically to wild type human serum albumin (UniProt Accession No. Q56G89). The amino acid sequence for wild type human serum albumin is shown in SEQ ID No. 9.

Human serum albumin (HSA, 2BXN) comprises approximately 60% of the plasma protein. HSA consists of a single chain, 585 amino acids in length, which incorporates three homologous domains (I, II, and III). Domain I consists of residues 5-197, domain II includes residues 198-382, and domain III is formed from residues 383-569. Each domain is comprised of two sub-domains termed A and B (IA; residues 5-107, IIA; residues 108-197, IIA; residues 198-296, IIB; residues 297-382, IIIA; residues 383-494, IIIB; residues 495-569).

A single domain antibody (sdAb), immunoglobulin single variable domain or protein of the invention “which binds” or is “capable of binding” an antigen of interest, e.g. human serum albumin, is one that binds the antigen with sufficient affinity such that the single domain antibody is useful as a therapeutic agent in targeting a cell or tissue expressing the antigen human serum albumin as described herein.

A single domain antibody, immunoglobulin single variable domain or protein described herein, binds specifically to human serum albumin. In other words, binding to the human serum albumin antigen is measurably different from a non-specific interaction. As demonstrated in the examples, the single domain antibodies do not cross react with mouse human serum albumin. Preferably, the single domain antibodies bind to human serum albumin and also bind to monkey serum albumin as shown in the examples.

The term “antibody” as used herein broadly refers to any immunoglobulin (Ig) molecule, or antigen binding portion thereof, comprised of four polypeptide chains, two heavy (H) chains and two light (L) chains, or any functional fragment, mutant, variant, or derivation thereof, which retains the essential epitope binding features of an Ig molecule.

In a full-length antibody, each heavy chain is comprised of a heavy chain variable region or domain (abbreviated herein as HCVR) and a heavy chain constant region. The heavy chain constant region is comprised of three domains, C_{H}1, C_{H}2 and C_{H}3. Each light chain is comprised of a light chain variable region or domain (abbreviated herein as LCVR) and a light chain constant region. The light chain constant region is comprised of one domain, C_{L}.

The heavy chain and light chain variable regions can be further subdivided into regions of hypervariability, termed complementarity determining regions (CDR), interspersed with regions that are more conserved, termed framework regions (FR). Each heavy chain and light chain variable region is composed of three CDRs and four FRs, arranged from amino-terminus to carboxy-terminus in the following order: FR1, CDR1, FR2, CDR2, FR3, CDR3, FR4.

Immunoglobulin molecules can be of any type (e.g., IgG, IgE, IgM, IgD, IgA and IgY), class (e.g., IgG 1, IgG2, IgG 3, IgG4, IgA1 and IgA2) or subclass. The term “CDR” refers to the complementarity-determining region within antibody variable sequences. There are three CDRs in each of the variable regions of the heavy chain and the light chain, which are designated CDR1, CDR2 and CDR3, for each of the variable regions. The term “CDR set” refers to a group of three CDRs that occur in a single variable region capable of binding the antigen. The exact boundaries of these CDRs can be defined differently according to different systems known in the art.

The Kabat Complementarity Determining Regions (CDRs) are based on sequence variability and are the most commonly used (Kabat et al., (1971) Ann. NY Acad. Sci. 190:382-391 and Kabat, et al., (1991) Sequences of Proteins of Immunological Interest, Fifth Edition, U.S. Department of Health and Human Services, NIH Publication No. 91-3242). Chothia refers instead to the location of the structural loops (Chothia and Lesk J. Mol. Biol. 196:901-917 (1987)). The Kabat numbering system is generally used when referring to a residue in the variable domain (approximately residues 1-107 of the light chain and residues 1-113 of the heavy chain). Another system is the ImMunoGeneTics (IMGT) numbering scheme. The IMGT numbering scheme is described in Lefranc et al., Dev. Comp. Immunol., 29, 185-203 (2005).

The system described by Kabat is used herein. The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion.

The term “antigen binding site” refers to the part of the antibody or antibody fragment that comprises the area that specifically binds to an antigen. An antigen binding site may be provided by one or more antibody variable domains. An antigen binding site is typically comprised within the associated V_{H} and V_{L} of an antibody or antibody fragment.

An antibody fragment is a portion of an antibody, for example as F(ab′)2, Fab, Fv, scFv, heavy chain, light chain, variable heavy (V_{H}), variable light (V_{L}) chain domain and the like. Functional fragments of a full length antibody retain the target specificity of a full antibody. Recombinant functional antibody fragments, such as Fab (Fragment, antibody), scFv (single chain variable chain fragments) and single domain antibodies (dAbs) have therefore been used to develop therapeutics as an alternative to therapeutics based on mAbs.

scFv fragments (˜25 kDa) consist of the two variable domains, V_{H} and V_{L}. Naturally, V_{H} and V_{L} domains are non-covalently associated via hydrophobic interactions and tend to dissociate. However, stable fragments can be engineered by linking the domains with a hydrophilic flexible linker to create a single chain Fv (scFv).

The smallest antigen binding fragment is the single variable fragment, namely the variable heavy (V_{H}) or variable light (V_{L}) chain domain. V_{H} and V_{L} domains respectively are capable of binding to an antigen. Binding to a light chain/heavy chain partner respectively or indeed the presence of other parts of the full antibody is not required for target binding. The antigen-binding entity of an antibody, reduced in size to one single domain (corresponding to the V_{H} or V_{L} domain), is generally referred to as a “single domain antibody” or “immunoglobulin single variable domain”. A single domain antibody (˜12 to 15 kDa) has thus either the V_{H} or V_{L} domain, i.e. it does not have other parts of a full antibody. Single domain antibodies derived from camelid heavy chain only antibodies that are naturally devoid of light chains as well as single domain antibodies that have a human heavy chain domain have been described. Antigen binding single V_{H} domains have also been identified from, for example, a library of murine V_{H} genes amplified from genomic DNA from the spleens of immunized mice and expressed in E. coli (Ward et al., 1989, Nature 341: 544-546). Ward et al. named the isolated single V_{H} domains “dAbs,” for “domain antibodies.” The term “dAb” generally refers to a single immunoglobulin variable domain (V_{H}, V_{HH} or V_{L}) polypeptide that specifically binds antigen. For use in therapy, human single domain antibodies are preferred over camelid derived V_{HH}, primarily because they are not as likely to provoke an immune response when administered to a patient.

The terms “single domain antibody, single variable domain or immunoglobulin single variable domain (ISV)” are all well known in the art and describe the single variable fragment of an antibody that binds to a target antigen. These terms are used interchangeably herein. A “single heavy chain domain antibody, single variable heavy chain domain, immunoglobulin single heavy chain variable domain (ISV), human V_{H} single domain” describes the single heavy chain variable fragment of an antibody which retains binding specificity to the antigen in the absence of light chain or other antibody fragments. A single variable heavy chain domain antibody does not comprise any other chains of a full length antibody; it does not have any light chains or constant domains. Thus, it is capable of binding to an antigen in the absence of light chain.

In one aspect, the invention relates to immunoglobulin single variable domains that bind human serum albumin. As explained below, the embodiments relate to single variable heavy chain domain antibodies/immunoglobulin single variable heavy chain domains which bind a HSA antigen. Thus, the single variable heavy chain domain antibody is capable of binding to HSA in the absence of light chain. Human single variable heavy chain domain antibodies (“V_{H} single domain antibody/single V_{H} domain antibody”) are particularly preferred. Such binding molecules are also termed Humabody® herein. Humabody® is a registered trademark of Crescendo Biologics Ltd.

Thus, in some embodiments, the isolated binding agents/molecules comprise or consist of at least one single domain antibody wherein said domain is a human immunoglobulin variable heavy chain domain; they are devoid of V_{L} domains or other antibody fragments and bind to the target antigen.

The term “isolated” refers to a moiety that is isolated form its natural environment. For example, the term “isolated” refers to a single domain antibody that is substantially free of other single domain antibodies, antibodies or antibody fragments. Moreover, an isolated single domain antibody may be substantially free of other cellular material and/or chemicals.

Each V_{H} domain antibody comprises three CDRs and four FRs, arranged from amino-terminus to carboxy-terminus in the following order: FR1-CDR1-FR2-CDR2-FR3-CDR3-FR4. Thus, in one embodiment of the invention, the domain is a human variable heavy chain (V_{H}) domain with the following formula FR1-CDR1-FR2-CDR2-FR3-CDR3-FR4.

Modifications to the C or N-terminal V_{H} framework sequence may be made to the single domain antibodies of the invention to improve their properties. For example, the V_{H} domain may comprise C- or N-terminal extensions. C-terminal extensions can be added to the C-terminal end of a V_{H} domain which terminates with the residues VTVSS (SEQ ID No. 10).

In one embodiment, the single domain antibodies of the invention comprise C-terminal extensions of from 1 to 50 residues, for example 1 to 10, e.g. 1, 2, 3, 4, 5, 6, 7, 8, 9 or 10, 1-20, 1-30 or 1-40 additional amino acids. In one embodiment, the single domain antibodies of the invention comprise additional amino acids of the human C_{H}1 domain thus that the C terminal end extends into the C_{H}1 domain. For example, C-terminal extensions may comprise neutral, nonpolar amino acids, such as A, L, V, P, M, G, I, F or W or neutral polar amino acids, such as S or T. C-terminal extensions may also be selected from peptide linkers or tags. Additionally, C or N-terminal residues can be peptide linkers that are for example used to conjugate the single domain antibodies of the invention to another moiety, or tags that aid the detection of the molecule. Such tags are well known in the art and include for, example linker His tags, e.g., hexa-His (HHHHHH, SEQ ID No. 11) or myc tags.

As used herein, the term “homology” or “identity” generally refers to the percentage of amino acid residues in a sequence that are identical with the residues of the reference polypeptide with which it is compared, after aligning the sequences and in some embodiments after introducing gaps, if necessary, to achieve the maximum percent homology, and not considering any conservative substitutions as part of the sequence identity. Thus, the percent homology between two amino acid sequences is equivalent to the percent identity between the two sequences. Neither N- or C-terminal extensions, tags or insertions shall be construed as reducing identity or homology. Methods and computer programs for the alignment are well known. The percent identity between two amino acid sequences can be determined using well known mathematical algorithms.

The variable domain of the single domain antibodies as described herein is a fully human or substantially fully human. The term V_{H} domain antibody as used herein designates a single human variable heavy chain domain antibody (as opposed to V_{HH} which designates a camelid heavy chain domain). As used herein, a human V_{H} domain includes a fully human or substantially fully human V_{H} domain. As used herein, the term human V_{H} domain also includes V_{H} domains that are isolated from heavy chain only antibodies made by transgenic mice expressing fully human immunoglobulin heavy chain loci, in particular in response to an immunisation with an antigen of interest (i.e. HSA), for example as described in WO2016/062990 and in the examples below. In one embodiment, a human V_{H} domain can also include a V_{H} domain that is derived from or based on a human V_{H} domain amino acid or produced from a human V_{H} nucleic acid sequence. Thus, the term human V_{H} domain includes variable heavy chain regions derived from or encoded by human immunoglobulin sequences and for example obtained from heavy chain only antibodies produced in transgenic mice expressing fully human unrearranged V, D, J gene segments. In some embodiments, a substantially human V_{H} domain or a V_{H} domain that is derived from or based on a human V_{H} domain may include amino acid residues not encoded by human germline immunoglobulin sequences (e.g., mutations introduced in vitro, e.g. by random or site-specific mutagenesis, or introduced by somatic mutation in vivo). The term “human V_{H} domain” therefore also includes a substantially human V_{H} domain wherein one or more amino acid residue has been modified, for example to remove sequence liabilities. For example, a substantially human V_{H} domain may include up to 10, for example 1, 2, 3, 4, 5, 6, 7, 8, 9 or 10 or up to 20 amino acid modifications compared to a germline human sequence.

However, the term “human V_{H} domain” or “substantially human V_{H} domain”, as used herein, is not intended to include antibodies in which CDR sequences derived from the germline of another mammalian species, such as a mouse, have been grafted onto human framework sequences. In one embodiment, the term “human V_{H} domain”, as used herein, is also not intended to include camelized V_{H} domains, that is human V_{H} domains that have been specifically modified, for example in vitro by conventional mutagenesis methods to select predetermined positions in the V_{H} domains sequence and introduce one or more point mutation at the predetermined position to change one or more predetermined residue to a specific residue that can be found in a camelid V_{HH} domain.

The molecules of the invention are advantageous because they are fully human and are thus not immunogenic. They do not require humanisation.

In a first aspect, there is provided a immunoglobulin single variable domain antibody that comprises

a) a CDR1 having SEQ ID NO. 2 or an amino acid sequence that has 1, 2, 3, 4, 5 or 5 differences with SEQ ID NO. 2

b) a CDR2 having SEQ ID NO. 3 or an amino acid sequence that has 1, 2, 3, 4, 5, 7, 8, 9, 10, 11, 12, 13, 14, 15, 16 or 17 differences with SEQ ID NO. 3 and/or

c) a CDR3 having SEQ ID NO. 4 or an amino acid sequence that has 1, 2, 3, 4, 5, 6, 7, 8, 9, 10, 11, 12, 13 or 14 differences with SEQ ID NO. 4.

In one embodiment, the immunoglobulin single variable domain has one of the CDRs defined above, e.g. CDR1, CDR2 or CDR3. In on embodiment the CDR is selected from SEQ ID NO. 2, 3 or 4 respectively. In another embodiment, the CDR is a variant and has substitutions as defined above. In another embodiment, one for two of the CDR sequences is as defined from SEQ ID

NO. 2, 3 or 4 and the remaining CDR is a variant of the respective CDR sequence 2, 3, or as applicable.

In one embodiment, the single variable domain antibody comprises or consists of SEQ ID NO. 1 or a sequence with at least 80%, 90% or 95% homology thereto.

SEQ ID NO. 1 is shown below:

(SEQ ID NO. 1, also termed Humabody® 1 herein)
EVQLLESGGG LVKPGGSLRL SCAASGFTFS NYNMNWVRQA
PGKRLEWVSS ISSAGTHIYS ADSVKGRFTI SRDNAKNSLY
LQMNSLRAED TGVYYCARDP HSTGWYKDFD YWGQGTLVTV
SS

The sequence for CDR1, CDR2 and CDR3 respectively is shown in bold above. The CDRs have the following sequence:

(SEQ ID NO. 2)
NYNMN CDR1:
(SEQ ID NO. 3)
SISSAGTHIYSADSVKG CDR2:
(SEQ ID NO. 4)
DPHSTGWYKDFDY CDR3:

In one embodiment, there is provided a single variable domain antibody that is capable of binding to human serum albumin and has 4 framework regions, FR1 to FR4 respectively, and 3 complementarity determining regions, CDR1 to CDR3 respectively, in which:

(i) CDR1 comprises or is the amino acid sequence as shown in SEQ ID NO. 2; CDR2 comprises or is the amino acid sequence SEQ ID NO. 3; and CDR3 comprises or is the amino acid sequence SEQ ID NO. 4 and wherein

(ii) the amino acid sequence has at least at least 85%, 90% or 95%, sequence identity the amino acid sequences of SEQ ID NO. 1.

In another aspect, there is provided a immunoglobulin single variable domain antibody that comprises

a) a CDR1 having SEQ ID NO. 6 or an amino acid sequence that has 1, 2, 3, 4, 5 or 5 differences with SEQ ID NO. 6

b) a CDR2 having SEQ ID NO. 7 or an amino acid sequence that has 1, 2, 3, 4, 5, 7, 8, 9, 10, 11, 12, 13, 14, 15, 16 or 17 differences with SEQ ID NO. 7 and/or

c) a CDR3 having SEQ ID NO. 8 or an amino acid sequence that has 1, 2, 3, 4, 5, 6, 7, 8, 9, 10, 11, 12, 13 or 14 differences with SEQ ID NO. 8.

In one embodiment, the immunoglobulin single variable domain has one of the CDRs defined above, e.g. CDR1, CDR2 or CDR3. In on embodiment the CDR is selected from SEQ ID NO. 6, 7 or 8 respectively. In another embodiment, the CDR is a variant and has substitutions as defined above. In another embodiment, one for two of the CDR sequences is as defined from SEQ ID NO. 6, 7 or 8 and the remaining CDR is a variant of the respective CDR sequence 6, 7, 8 or as applicable.

In one embodiment, the single variable domain antibody comprises or consists of SEQ ID NO. 5 or a sequence with at least 80%, 90% or 95% homology thereto.

SEQ ID NO. 5 is shown below:

(SEQ ID NO. 5, also termed Humabody® 2 herein)
EVQLLESGGG LVKPGGSLRL SCAASGFTVS SYTMNWVRQA
PGKGLEWVSS ISSSGRYIYY ADSVKGRFTI SRDNAKNSLY
LQMNSLRAED TAVYYCARDP RMVGNPHEFD IWGQGTMVTV
SS

The sequence for CDR1, CDR2 and CDR3 respectively is shown in bold above. The CDRs have the following sequence:

(SEQ ID NO. 6)
SYTMN CDR1:
(SEQ ID NO. 7)
SISSSGRYIYYADSVKG CDR2:
(SEQ ID NO. 8)
DPRMVGNPHEFDI CDR3:

In one embodiment, there is provided a single variable domain antibody that is capable of binding to human serum albumin and has 4 framework regions, FR1 to FR4 respectively, and 3 complementarity determining regions, CDR1 to CDR3 respectively, in which:

(i) CDR1 comprises or is the amino acid sequence as shown in SEQ ID NO. 6; CDR2 comprises or is the amino acid sequence SEQ ID NO. 7; and CDR3 comprises or is the amino acid sequence SEQ ID NO. 8 and wherein

(ii) the amino acid sequence has at least at least 85%, 90% or 95%, sequence identity the amino acid sequences of SEQ ID NO. 5.

Sequence homology/identity as used above can be at least 80%, 81%, 82%, 83%, 84%, 85%, 86%, 87%, 88% 89%, 90%, 91%, 92%, 93%, 94%, 95%, 96%, 97%, 98% or 99% for example at least 95%, 96%, 97%, 98% or 99% sequence homology/identity.

The immunoglobulin single variable domain antibody may be a variant of SEQ ID NO.1 or SEQ ID NO. 5 having one or more amino acid substitutions, deletions, insertions or other modifications, and which retains a biological function of the single domain antibody, that is binding to HSA. Thus, variant V_{H} single domain antibody can be sequence engineered. Modifications may include one or more substitution, deletion or insertion of one or more codons encoding the single domain antibody or polypeptide that results in a change in the amino acid sequence as compared with the native sequence V_{H} single domain antibody or polypeptide. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 25, for example 1 to 5, 1 to 10, 1 to 15, 1 to 20 amino acids, for example 1, 2, 3, 4, 5, 6, 7, 8, 9 or 10 amino acids. The variation allowed may be determined by systematically making insertions, deletions or substitutions of amino acids in the sequence and testing the resulting variants for activity exhibited by the full-length or mature native sequence. A variant of a V_{H} single domain antibody described herein has at least 90%, 91%, 92%, 93%, 94%, 95%, 96%, 97%, 98% or 99% sequence homology/identity to the non-variant molecule. In one embodiment, the modification is a conservative sequence modification. As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an sdAb of the invention by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of a single domain antibody of the invention can be replaced with other amino acid residues from the same side chain family and the altered antibody can be tested for retained function (i.e., HSA binding) using the functional assays described herein.

Thus, these amino acid changes can typically be made without altering the biological activity, function, or other desired property of the polypeptide, such as its affinity or its specificity for antigen. In general, single amino acid substitutions in nonessential regions of a polypeptide do not substantially alter biological activity. Furthermore, substitutions of amino acids that are similar in structure or function are less likely to disrupt the polypeptides' biological activity. Abbreviations for the amino acid residues that comprise polypeptides and peptides described herein, and conservative substitutions for these amino acid residues are shown in Table 1 below.

TABLE 1
Amino Acid Residues and Examples of Conservative Amino
Acid Substitutions

| Original residue | |
|---|---|
| Three letter code, single letter code | Conservative substitution |
| Alanine, Ala, A | Gly, Ser |
| Arginine, Arg, R | Lys, His |
| Asparagine, Asn, N | Gln, His |
| Aspartic acid Asp, D | Glu, Asn |
| Cysteine, Cys, C | Ser, Ala |
| Glutamine, Gln, Q | Asn |
| Glutamic acid, Glu, E | Asp, Gln |
| Glycine, Gly, G | Ala |
| Histidein, His, H | Asn, Gln |
| Isoleucine, Ile, I | Leu, Val |
| Leucine, Leu, L | Ile, Val |
| Lysine, lys, K | Ar, His |
| Methionine, Met, M | Leu, Ile, Tyr |
| Phenylalanine, Phe, F | Tyr, Met, Leu |
| Proline, Pro, P | Ala |
| Serine, Ser, S | Thr |
| Threonine, Thr, T | Ser |
| Tryptophan, Trp, W | Tyr, Phe |
| Tyrosine, Tyr, Y | Try, Phe |
| Valine, Val, V | Ile, Leu |

In some embodiments, the invention provides a V_{H} single domain antibody that is a variant of a V_{H} single domain antibody compared to SEQ ID NO. 1, SEQ ID NO. 30 or SEQ ID NO. 5 that comprises one or more sequence modification and has improvements in one or more of a property such as binding affinity, specificity, thermostability, expression level, effector function, glycosylation, reduced immunogenicity, or solubility as compared to the unmodified single domain antibody.

A skilled person will know that there are different ways to identify, obtain and optimise the antigen binding molecules as described herein, including in vitro and in vivo expression libraries. This is further described in the examples. Optimisation techniques known in the art, such as display (e.g., ribosome and/or phage display) and/or mutagenesis (e.g., error-prone mutagenesis) can be used. The invention therefore also comprises sequence optimised variants of the single domain antibodies described herein.

In one embodiment, modifications can be made to decrease the immunogenicity of the single domain antibody. For example, one approach is to revert one or more framework residues to the corresponding human germline sequence. More specifically, a single domain antibody that has undergone somatic mutation may contain framework residues that differ from the germline sequence from which the single domain antibody is derived. Such residues can be identified by comparing the single domain antibody framework sequences to the germline sequences from which the single domain antibody is derived. In one embodiment, all framework sequences are germline sequence.

To return one or more of the amino acid residues in the framework region sequences to their germline configuration, the somatic mutations can be “backmutated” to the germline sequence by, for example, site-directed mutagenesis or PCR-mediated mutagenesis.

Another type of framework modification involves mutating one or more residues within the framework region, or even within one or more CDR regions, to remove T cell epitopes to thereby reduce the potential immunogenicity of the antibody.

In still another embodiment, glycosylation is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for the antigen.

In one embodiment, the one or more substitution is in the CDR1, 2 or 3 region. For example, there may be 1, 2, 3, 4, 5 or more amino acid substitutions in the CDR1, 2 or 3. In another example, there may be 1 or 2 amino acid deletions. In one embodiment, the one or more substitution is in the framework region. For example, there may be 1 to 10 or more amino acid substitutions in the framework region.

In one embodiment, the variant comprises substitutions at one or more of the following positions one or more of the following substitutions with reference to SEQ ID NO. 1 or combinations thereof: E1, V5, G44, Y60, 92A, 28T-A N31, N33, A54, G55, H57, 158 and/or Y106. Positions are according to Kabat.

In one embodiment, the variant comprises substitutions at one or more of the following positions one or more of the following substitutions with reference to SEQ ID NO. 1 or combinations thereof such as at positions: E1→Q; 5V-L; G44→R; Y60→S; 92A→G; 28T→A; N31→S; N33→T; A54→G; G55→S; H57→Y; I58→K and/or Y106→F. Positions are according to Kabat.

In one embodiment, the variant comprises 1, 2, 3, 3, 4, 5, 6, 7, 8, 9, 10, 11, 12 or 13 of the modifications listed above. Combinations of the modifications are thus specifically envisaged.

In one embodiment, the variant comprises one or more of the following substitutions with reference to SEQ ID NO. 5 or combinations thereof, such as at positions: V5, T28, N84, S50, S54, G55, R56, Y60, L103, El 08 and/or 1111. Positions are according to Kabat.

One variant of SEQ ID NO.1 according to the invention is shown in SEQ ID No. 30 below. EVQLVESGGG LVKPGGSLRL SCAASGFTFS NYNMNWVRQA PGKGLEWVSS ISSAGTHIYY A DSVKGRFTI SRDNAKNSLY LQMNSLRAED TAVYYCARDP HSTGWYKDFD YWGQGTLVTVSS

The corresponding nucleic acid is shown below (SEQ ID NO. 31).

GAGGTGCAGC TGGTGGAGTC TGGGGGAGGC CTGGTCAAGC
CTGGGGGGTC CCTGAGACTC TCCTGTGCAG CCTCTGGATT
CACCTTCAGT AACTATAACA TGAACTGGGT CCGCCAGGCT
CCAGGGAAGG GGCTGGAGTG GGTCTCATCG ATTAGTAGTG
CTGGTACTCA CATATACTAC GCAGACTCAG TGAAGGGCCG
ATTCACCATC TCCAGAGACA ACGCCAAGAA CTCACTGTAT
CTGCAAATGA ACAGCCTGAG AGCCGAGGAC ACAGCTGTTT
ATTACTGTGC GAGAGATCCT CATAGCACTG GCTGGTACAA
GGACTTTGAC TACTGGGGCC AGGGAACCCT GGTCACCGTC
TCCTCA

In one embodiment, the variant comprises one or more of the following substitutions with reference to SEQ ID NO. 5 or combinations thereof, such as at positions: V5→L; T28→N or A; N84→H; S50→A; S54→N; G55→S; R56→T; Y60→H; L103→; E108→A and/or I111→V. Positions are according to Kabat.

As mentioned, the amino acid sequences provided by the invention are proteins that can bind to, and that can in particular specifically (as described herein) bind to, human serum albumin. Thus, they can be used as binding units or binding domains for binding to human serum albumin, for example to confer an increase in half-life (as defined herein) to therapeutic compounds, moieties or entities.

The term “half-life” as used can generally refer to the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of an amino acid sequence, compound or polypeptide of the invention can be determined in any manner known per se, such as by pharmacokinetic analysis. Suitable techniques will be clear to the person skilled in the art. The half-life can be expressed using parameters such as the t½-alpha, t½-beta and the area under the curve (AUC). Half-lives (t alpha and t beta) and AUC can be determined from a curve of serum concentration of conjugate or fusion against time. Thus, the term “half-life” as used herein in particular refers to the t½-beta or terminal half-life (in which the t½-alpha and/or the AUC or both may be kept out of considerations).

For example, in a first phase (the alpha phase) the drug composition (e. g., drug conjugate, noncovalent drug conjugate, drug fusion) is undergoing mainly distribution in the patient, with some elimination. A second phase (beta phase) is the terminal phase when the drug composition (e. g., drug conjugate, noncovalent drug conjugate, drug fusion) has been distributed and the serum concentration is decreasing as the drug composition is cleared from the patient. The t alpha half-life is the half-life of the first phase and the t beta half-life is the half-life of the second phase.

The HSA binding immunoglobulin variable domain of the invention:
Can have a serum half life in man (expressed as t½) of 1 to 72 hours, for example 10 or more, for example up to 20 hours or 12, 24, 36 or 48 hours and/or
When linked to a therapeutic moiety confers to the resulting protein a serum half life in man that is 1 to 72 hours, for example 10 or more, for example up to 20 hours or 12, 24, 36 or 48 hours.

In one embodiment, the HSA binding immunoglobulin variable domain extends the half life of a molecule by about 20 to 78 hours in the genOway® HSA/FcRn mouse model as shown in the examples.

In one embodiment, the HSA binding immunoglobulin variable domain confers a half life to a molecule of about 84 hours in cynomolgus macaque. This can be as shown in the examples, for example for HSA binder of SEQ ID NO:30.

The HSA binding immunoglobulin variable domains of the invention also have excellent storage stability as shown in example 9. They are particularly suited to extending the half life of V_{H} single domain antibodies.

The invention also relates to binding molecules that comprise an immunoglobulin single variable domain antibody described herein, for example comprising or consisting of SEQ ID NO. 1, SEQ ID NO. 30 or SEQ ID NO. 5 or and a second moiety. In one embodiment, the moiety is a therapeutic moiety. The binding molecule can be polypeptide, protein or construct. Provided are thus also a fusion protein, multivalent and multispecific proteins or constructs comprising a single variable domain antibody described herein. immunoglobulin single variable domain antibody described herein, for example comprising or consisting of SEQ ID NO. 1, SEQ ID NO. 30 or SEQ ID NO. 5 is for use with a moiety that binds to another target, such as a therapeutic target.

In one embodiment, the therapeutic moiety is a binding molecule, for example selected from an antibody or antibody fragment (e.g., a Fab, F(ab′)2, Fv, a single chain Fv fragment (scFv) or single domain antibody, for example a V_{H} or V_{HH} domain) or antibody mimetic protein. In one embodiment, the single domain antibody of the invention can be linked to an antibody Fc region or fragment thereof, comprising one or both of C_{H}2 and C_{H}3 domains, and optionally a hinge region. In one embodiment, the at least second moiety is a V_{H} domain.

In one embodiment, the proteins or polypeptides that comprise the immunoglobulin single variable domain that binds to HSA as described herein and a second moiety are fusion proteins.

In one embodiment, the proteins or polypeptides that comprise the immunoglobulin single variable domain that binds to HSA as described herein and a second moiety are drug conjugates.

As used herein “conjugate” refers to a composition comprising an antigen-binding fragment of an antibody that binds serum albumin that is bonded to a drug.

Such conjugates include “drug conjugates” which comprise an antigen-binding fragment of an antibody that binds serum albumin to which a drug is covalently bonded, and “non-covalent drug conjugates” which comprise an antigen-binding fragment of an antibody that binds serum albumin to which a drug is noncovalently bonded.

As used herein, “drug conjugate” refers to a composition comprising an antigen-binding fragment of an antibody that binds serum albumin to which a drug is covalently bonded. The drug can be covalently bonded to the antigen-binding fragment directly or indirectly through a suitable linker or spacer moiety. The drug can be bonded to the antigen-binding fragment at any suitable position, such as the amino-terminus, the carboxyl-terminus or through suitable amino acid side chains.

In one embodiment, the immunoglobulin single variable domain is linked to the second moiety with a peptide linker or other suitable linker to connect the two moieties.

The term “peptide linker” refers to a peptide comprising one or more amino acids. A peptide linker comprises 1 to 50, for example 1 to 20 amino acids. Peptide linkers are known in the art and non-limiting examples are described herein. Suitable, non-immunogenic linker peptides are, for example, linkers that include G and/or S residues, (G4S)n, (SG4)n or G4(SG4)n peptide linkers, wherein “n” is generally a number between 1 and 10, e.g., 1, 2, 3, 4, 5, 6, 7, 8, 9 or 10. In one embodiment, the peptide is for example selected from the group consisting of GGGGS (SEQ ID NO:12), GGGGSGGGGS (SEQ ID NO:13), SGGGGSGGGG (SEQ ID NO:14), GGGGSGGGGSGGGG (SEQ ID NO:15), GSGSGSGS (SEQ ID NO:16), GGSGSGSG (SEQ ID NO:17), GGSGSG (SEQ ID NO:18) and GGSG (SEQ ID NO:19).

The binding agent may be multispecific, for example bispecific. In one embodiment, the binding molecule comprises a first V_{H} single domain antibody that binds to HSA as described herein (V_{H} (A)) and a second V_{H} single domain antibody (V_{H} (B)) that binds to another antigen and thus has the following formula: V_{H} (A)-L-V_{H} (B). V_{H} (A) is conjugated to V_{H} (B), that is linked to V_{H} (B), for example with a peptide linker. L denotes a linker.

Each V_{H} comprises CDR and FR regions. Thus, the binding molecule may have the following formula: FR1 (A)-CDR1 (A)-FR2(A)-CDR2(A)-FR3(A)-CDR3(A)-FR4(A)-L-FR1(B)-CDR1(B)-FR2(B)-CDR2(B)-FR3(B)-CDR3(B)-FR4(B).

The order of the single V_{H} domains A and B is not particularly limited, so that, within a polypeptide of the invention, single variable domain A may be located N-terminally and single variable domain B may be located C-terminally, or vice versa.

In one embodiment, the binding molecule is bispecific. Thus, in one aspect, the invention relates to a bispecific molecule comprising a single domain antibody described herein linked to a second functional moiety having a different binding specificity than said single domain antibody.

In one embodiment the binding molecule, e.g. the protein or construct is multispecific and comprises a further, i.e. third, fourth, fifth etc moiety.

In one embodiment of the multispecific protein, the HSA binding V_{H} domain is located at the C terminus of the protein. In one embodiment of the multispecific protein, the HSA binding V_{H} domain is located at the N terminus of the protein. In one embodiment of the multispecific protein, the HSA binding V_{H} domain is not located terminally.

The second or further therapeutic moiety can be selected from a moiety that binds for example a tumor antigen or an immunooncology target, but a skilled person would know that the invention is not thus limited.

The invention also relates to the use of an immunoglobulin single variable domain as described herein extending the half life of a therapeutic moiety when said an immunoglobulin single variable domain according to any of claims is linked to said therapeutic moiety in a fusion protein.

The invention also relates to the use of an immunoglobulin single variable domain as described herein extending the half life of a therapeutic moiety when said an immunoglobulin single variable domain according to any of claims is linked to said therapeutic moiety in a fusion protein. For example, it can be used to extend the half life of a protein comprising a sdAb that binds to CD137 and an sdAb that binds to PSMA or PD-1, for example as described herein.

The immunoglobulin single variable domain as described herein, for example the molecule may be in the format of V_{H} (A)-V_{H} (B)-V_{H} (C), V_{H} (B)-V_{H} (A)-V_{H} (C), V_{H} (C)-V_{H} (A)-V_{H}(B) or V_{H} (C)-V_{H}(B)-V_{H} (A) wherein A is a sdAb that binds to CD137, B an sdAb that binds to PSMA or PD-1 and C is immunoglobulin single variable domain as described herein (e.g. SEQ ID NO. 1, SEQ ID NO. 30 or SEQ ID NO. 5). The order of the V_{H} is flexible and as explained elsewhere, suitable linkers connect the V_{H} molecules. Exemplary molecules comprise or consist of a sequence selected from SEQ ID Nos. 22, 23, 26, 28, 33, 34 or 35.

In one embodiment, the single variable heavy chain domain antibody is obtained or obtainable from a transgenic rodent that expresses a transgene comprising unrearranged human V, D and J regions, in particular a rodent that produces human heavy chain only antibodies. In one embodiment, the said rodent does not produce functional endogenous light and heavy chains.

Generally, unless indicated otherwise herein, the immunoglobulin single variable domain, polypeptides, proteins and other compounds and constructs referred to herein will be intended for use in prophylaxis or treatment of diseases or disorders in man (and/or optionally also in warm-blooded animals and in particular mammals). Thus, generally, the immunoglobulin single variable domain, polypeptides, proteins and other compounds and constructs described herein are preferably such that they can be used as, and/or can suitably be a part of, a (biological) drug or other pharmaceutically or therapeutically active compound and/or of a pharmaceutical product or composition.

Thus, the invention also relates to a pharmaceutical composition or formulation comprising an immunoglobulin single variable domain polypeptide, protein or construct as described herein, e.g. a binding molecule or fusion protein that comprises the HSA-binding single domain as described herein. The pharmaceutical composition may optionally comprise a pharmaceutically acceptable carrier. Immunoglobulin single variable domain polypeptide, protein or construct or the pharmaceutical composition can be administered by any convenient route, including but not limited to oral, topical, parenteral, sublingual, rectal, vaginal, ocular, intranasal, pulmonary, intradermal, intravitreal, intramuscular, intraperitoneal, intravenous, subcutaneous, intracerebral, transdermal, transmucosal, by inhalation, or topical, particularly to the ears, nose, eyes, or skin or by inhalation.

Parenteral administration includes, for example, intravenous, intramuscular, intraarterial, intraperitoneal, intranasal, rectal, intravesical, intradermal, topical or subcutaneous administration. Preferably, the compositions are administered parenterally.

The pharmaceutically acceptable carrier or vehicle can be particulate, so that the compositions are, for example, in tablet or powder form. The term “carrier” refers to a diluent, adjuvant or excipient, with which a drug antibody conjugate of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the single domain antibody of the present invention or compositions and pharmaceutically acceptable carriers are sterile. Water is a preferred carrier when the drug antibody conjugates of the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The pharmaceutical composition of the invention can be in the form of a liquid, e.g., a solution, emulsion or suspension. The liquid can be useful for delivery by injection, infusion (e.g., IV infusion) or sub-cutaneously. When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e. g. a gelatin capsule), it can contain, in addition to materials of the above type, a liquid carrier such as polyethylene glycol, cyclodextrin or a fatty oil.

The composition can be in the form of a liquid, e. g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units. In specific embodiments, it can be desirable to administer the composition locally to the area in need of treatment, or by intravenous injection or infusion.

The invention further extends to methods for the treatment of a disease, e.g. cancer, comprising administration of a pharmaceutical composition or formulation described herein or a binding molecule or fusion protein that comprises the HSA-binding single domain as described herein. Also envisaged is a pharmaceutical composition or formulation described herein or a binding molecule or fusion protein that comprises the HSA-binding single domain as described herein for use in the treatment of disease; e.g. for use in the treatment of cancer. Also envisaged is the use of a pharmaceutical composition or formulation described herein or a binding molecule or fusion protein that comprises the HSA-binding single domain as described herein fin the manufacture of a medicament for the treatment of cancer.

The amount of the therapeutic that is effective/active in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances. Factors like age, body weight, sex, diet, time of administration, rate of excretion, condition of the host, drug combinations, reaction sensitivities and severity of the disease shall be taken into account.

Typically, the amount is at least about 0.01% of a single domain antibody of the present invention by weight of the composition. When intended for oral administration, this amount can be varied to range from about 0.1% to about 80% by weight of the composition. Preferred oral compositions can comprise from about 4% to about 50% of the single domain antibody of the present invention by weight of the composition.

Preferred compositions of the present invention are prepared so that a parenteral dosage unit contains from about 0.01% to about 2% by weight of the single domain antibody of the present invention.

For administration by injection, the composition can comprise from about typically about 0.1 mg/kg to about 250 mg/kg of the subject's body weight, preferably, between about 0.1 mg/kg and about 20 mg/kg of the animal's body weight, and more preferably about 1 mg/kg to about 10 mg/kg of the animal's body weight. In one embodiment, the composition is administered at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks.

As used herein, “treat”, “treating” or “treatment” means inhibiting or relieving a disease or disorder. For example, treatment can include a postponement of development of the symptoms associated with a disease or disorder, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the mammals, e.g., human patients, being treated. Many medical treatments are effective for some, but not all, patients that undergo the treatment.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

The molecules or pharmaceutical composition of the invention may be administered as the sole active ingredient or in combination with one or more other therapeutic agent. A therapeutic agent is a compound or molecule which is useful in the treatment of a disease. Examples of therapeutic agents include antibodies, antibody fragments, drugs, toxins, nucleases, hormones, immunomodulators, pro-apoptotic agents, anti-angiogenic agents, boron compounds, photoactive agents or dyes and radioisotopes.

The invention also relates to a method for extending the half life of a protein comprising joining said protein to an immunoglobulin single variable domain as described herein.

The invention also relates to a nucleic acid sequence that encodes an amino acid sequence described herein. In one embodiment, said nucleic acid is SEQ ID NO. 20 or a nucleic acid having at least 90% sequence homology thereto. In one embodiment, said nucleic acid is SEQ ID NO. 21 or a nucleic acid having at least 90% sequence homology thereto. In one embodiment, said nucleic acid sequence is linked with a linker to a second nucleic acid sequence. In one embodiment, said second nucleic acid encodes a therapeutic moiety. In one embodiment, said linker is a nucleic acid linker.

SEQ ID NO. 20
GAGGTGCAGC TGTTGGAGTC TGGGGGAGGC CTGGTCAAGC
CTGGGGGGTC CCTGAGACTC TCCTGTGCAG CCTCTGGATT
CACCTTCAGT AACTATAACA TGAACTGGGT CCGCCAGGCT
CCAGGGAAGA GGCTGGAGTG GGTCTCATCG ATTAGTAGTG
CTGGTACTCA CATATACTCC GCAGACTCAG TGAAGGGCCG
ATTCACCATC TCCAGAGACAACGCCAAGAA CTCACTGTAT
CTGCAAATGA ACAGCCTGAG AGCCGAGGAC ACAGGTGTTT
ATTACTGTGC GAGAGATCCT CATAGCACTG GCTGGTACAA
GGACTTTGAC TACTGGGGCC AGGGAACCCT GGTCACCGTC
TCCTCA
SEQ ID NO. 21
GAGGTGCAGC TGTTGGAGTC TGGGGGAGGC CTGGTCAAGC
CGGGGGGGTC CCTGAGACTC TCCTGTGCAG CCTCTGGATT
CACCGTCAGT AGCTATACCA TGAACTGGGT CCGCCAGGCT
CCGGGGAAGG GGCTGGAGTG GGTCTCATCC ATTAGTAGTA
GTGGTCGTTA CATATACTAC GCAGACTCAG TGAAGGGCCG
ATTCACCATC TCCAGAGACA ACGCCAAGAA CTCATTATAT
CTGCAAATGA ACAGCCTGAG AGCCGAGGAC ACAGCTGTAT
ATTATTGTGC GAGAGATCCC CGTATGGTTG GGAACCCCCA
TGAATTTGAT ATCTGGGGCC AAGGGACAAT GGTCACCGTC
TCCTCA

The invention also relates to a vector comprising a nucleic acid sequence as described herein. The invention also relates to a host cell comprising the nucleic acid sequence as described herein or a vector as described herein. The host cell may be a mammalian, bacterial or yeast cell.

The invention also relates to a kit comprising an immunoglobulin single variable domain or pharmaceutical composition as described herein, a protein or construct as described herein or a pharmaceutical composition as described herein and optionally instructions for use.

A single domain antibody described herein can be obtained from a transgenic mammal, for example a rodent, that expresses heavy chain only antibodies upon stimulation with an HSA antigen. The transgenic rodent, for example a mouse, preferably has a reduced capacity to express endogenous antibody genes. Thus, in one embodiment, the rodent has a reduced capacity to express endogenous light and/or heavy chain antibody genes. The rodent may therefore comprise modifications to disrupt expression of endogenous kappa and lambda light and/or heavy chain antibody genes so that no functional light and/or heavy chains are produced, for example as further explained below.

One aspect also relates to a method for producing human heavy chain only antibodies or a binding molecule having a V_{H} domain as described herein capable of binding HSA said method comprising

a) immunising a transgenic rodent, e.g. a mouse, with an HSA antigen wherein said rodent expresses a nucleic acid construct comprising unrearranged human heavy chain V genes and is not capable of making functional endogenous light or heavy chains,

b) isolating human heavy chain only antibodies.

Further steps can include isolating a V_{H} domain from said heavy chain only antibody, for example by generating a library of sequences comprising V_{H} domain sequences from said rodent, e.g. a mouse and isolating sequences comprising V_{H} domain sequences from said libraries.

Another aspect also relates to a method for producing a single V_{H} domain antibody capable of binding human HSA said method comprising

a) immunising a transgenic rodent, e.g. a mouse. with an HSA antigen wherein said rodent expresses a nucleic acid construct comprising unrearranged human heavy chain V genes and is not capable of making functional endogenous light or heavy chains,

b) generating a library of sequences comprising V_{H} domain sequences from said rodent, e.g. a mouse and

c) isolating sequences comprising V_{H} domain sequences from said libraries.

Further steps may include identifying a single V_{H} domain antibody or heavy chain only antibody that binds to HSA, for example by using functional assays as shown in the examples.

Methods for preparing or generating the polypeptides, nucleic acids, host cells, products and compositions described herein using in vitro expression libraries can comprise the steps of: a) providing a set, collection or library of nucleic acid sequences encoding amino acid sequences; and

b) screening said set, collection or library for amino acid sequences that can bind to/have affinity for HSA and

c) isolating the amino acid sequence(s) that can bind to/have affinity for HSA.

Provided is also a method for preparing a single V_{H} domain antibody, binding molecule or fusion protein described herein which method comprises cultivating or maintaining a host cell as described herein under conditions such that said host cell produces or expresses a single V_{H} domain antibody, binding molecule or fusion protein described herein and optionally further comprises isolating the a single V_{H} domain antibody, binding molecule or fusion protein described herein so produced.

In the above method, the set, collection or library of amino acid sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art (see for example Phage Display of Peptides and Proteins: A Laboratory Manual, Academic Press; 1st edition (Oct. 28, 1996) Brian K. Kay, Jill Winter, John McCafferty). Libraries, for example phage libraries, are generated by isolating a cell or tissue expressing an antigen-specific, heavy chain-only antibody, cloning the sequence encoding the V_{H} domain(s) from mRNA derived from the isolated cell or tissue and displaying the encoded protein using a library. The V_{H} domain(s) can be expressed in bacterial, yeast or other expression systems.

In the various aspects and embodiments as out herein, the term rodent may relate to a mouse or a rat. In one embodiment, the rodent is a mouse. The mouse may comprise a non-functional endogenous lambda light chain locus. Thus, the mouse does not make a functional endogenous lambda light chain. In one embodiment, the lambda light chain locus is deleted in part or completely or rendered non-functional through insertion, inversion, a recombination event, gene editing or gene silencing. For example, at least the constant region genes C1, C2 and C3 may be deleted or rendered non-functional through insertion or other modification as described above. In one embodiment, the locus is functionally silenced so that the mouse does not make a functional lambda light chain.

Furthermore, the mouse may comprise a non-functional endogenous kappa light chain locus. Thus, the mouse does not make a functional endogenous kappa light chain. In one embodiment, the kappa light chain locus is deleted in part or completely or rendered non-functional through insertion, inversion, a recombination event, gene editing or gene silencing. In one embodiment, the locus is functionally silenced so that the mouse does not make a functional kappa light chain.

The mouse having functionally-silenced endogenous lambda and kappa L-chain loci may, for example, be made as disclosed in WO 2003/000737, which is hereby incorporated by reference in its entirety.

Furthermore, the mouse may comprise a non-functional endogenous heavy chain locus, for example as described in WO 2004/076618 (hereby incorporated by reference in its entirety). Thus, the mouse does not make a functional endogenous heavy chain. In one embodiment, the heavy chain locus is deleted in part or completely or rendered non-functional through insertion, inversion, a recombination event, gene editing or gene silencing. In one embodiment, the locus is functionally silenced so that the mouse does not make a functional heavy chain.

In one embodiment, the mouse comprises a non-functional endogenous heavy chain locus, a non-functional endogenous lambda light chain locus and a non-functional endogenous kappa light chain locus. The mouse therefore does not produce any functional endogenous light or heavy chains. Thus, the mouse is a triple knockout (TKO) mouse.

The transgenic mouse may comprise a vector, for example a Yeast Artificial Chromosome (YAC) for expressing a heterologous, preferably a human, heavy chain locus. YACs are vectors that can be employed for the cloning of very large DNA inserts in yeast. As well as comprising all three cis-acting structural elements essential for behaving like natural yeast chromosomes (an autonomously replicating sequence (ARS), a centromere (CEN) and two telomeres (TEL)), their capacity to accept large DNA inserts enables them to reach the minimum size (150 kb) required for chromosome-like stability and for fidelity of transmission in yeast cells. The construction and use of YACs is well known in the art (e.g., Bruschi, C. V. and Gjuracic, K. Yeast Artificial Chromosomes, Encyclopaedia of Life Sciences, 2002 Macmillan Publishers Ltd, Nature Publishing Group).

For example, the YAC may comprise a plethora of unrearranaged human V_{H}, D and J genes in combination with mouse immunoglobulin constant region genes lacking C_{H}1 domains, mouse enhancer and regulatory regions. The human V_{H}, D and J genes are human V_{H}, D and J loci and they are unrearranged genes that are fully human. The YAC may be as described in WO2016/062990.

Alternative methods known in the art may be used for deletion or inactivation of endogenous mouse or rat immunoglobulin genes and introduction of human V, D and J genes in combination with mouse immunoglobulin constant region genes lacking C_{H}1 domains, mouse enhancer and regulatory regions.

Transgenic mice can be created according to standard techniques as illustrated in the examples. The two most characterised routes for creating transgenic mice are via pronuclear microinjection of genetic material into freshly fertilised oocytes or via the introduction of stably transfected embryonic stem cells into morula or blastocyst stage embryos. Regardless of how the genetic material is introduced, the manipulated embryos are transferred to pseudo-pregnant female recipients where pregnancy continues and candidate transgenic pups are born.

The main differences between these broad methods are that ES clones can be screened extensively before their use to create a transgenic animal. In contrast, pronuclear microinjection relies on the genetic material integrating to the host genome after its introduction and, generally speaking, the successful incorporation of the transgene cannot be confirmed until after pups are born.

There are many methods known in the art to both assist with and determine whether successful integration of transgenes occurs. Transgenic animals can be generated by multiple means including random integration of the construct into the genome, site-specific integration, or homologous recombination. There are various tools and techniques that can be used to both drive and select for transgene integration and subsequent modification including the use of drug resistance markers (positive selection), recombinases, recombination-mediated cassette exchange, negative selection techniques, and nucleases to improve the efficiency of recombination. Most of these methods are commonly used in the modification of ES cells. However, some of the techniques may have utility for enhancing transgenesis mediated via pronuclear injection.

Further refinements can be used to give more efficient generation of the transgenic line within the desired background. As described above, in preferred embodiments, the endogenous mouse immunoglobulin expression is silenced to permit sole use of the introduced transgene for the expression of the heavy-chain only repertoire that can be exploited for drug discovery. Genetically-manipulated mice, for example TKO mice that are silenced for all endogenous immunoglobulin loci (mouse heavy chain, mouse kappa chain and mouse lambda chain) can be used as described above. The transfer of any introduced transgene to this TKO background can be achieved via breeding, either conventional or with the inclusion of an IVF step to give efficient scaling of the process. However, it is also possible to include the TKO background during the transgenesis procedure. For example, for microinjection, the oocytes may be derived from TKO donors. Similarly, ES cells from TKO embryos can be derived for use in transgenesis.

Triple knock-out mice into which transgenes have been introduced to express immunoglobulin loci are referred to herein as TKO/Tg.

In one embodiment, the mouse is as described in WO2016/062990. The invention also relates to a rodent, preferably a mouse which expresses a human heavy chain locus and which has been immunized with a HSA antigen. The invention also relates to a rodent as described above, preferably a mouse which expresses a heavy chain only antibody comprising a human V_{H} domain that binds to human HSA. Preferably, said rodent is not capable of making functional endogenous kappa and lambda light and/or heavy chains. The human heavy chain locus is located on a transgene which can be as described above.

The invention also relates to an anti-human HSA single V_{H} domain antibody or an anti-human HSA heavy chain only antibody comprising a human V_{H} domain or obtained or obtainable from a rodent, preferably a mouse, immunised with a human HSA antigen and which expresses a human heavy chain locus. Preferably, said rodent is not capable of making functional endogenous kappa and lambda light and/or heavy chains. The human heavy chain locus is located on a transgene which can be as described above.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present disclosure, including methods, as well as the best mode thereof, of making and using this disclosure, the following examples are provided to further enable those skilled in the art to practice this disclosure. However, those skilled in the art will appreciate that the specifics of these examples should not be read as limiting on the invention, the scope of which should be apprehended from the claims and equivalents thereof appended to this disclosure. Various further aspects and embodiments of the present disclosure will be apparent to those skilled in the art in view of the present disclosure.

All documents mentioned in this specification are incorporated herein by reference in their entirety, including references to gene accession numbers, scientific publications and references to patent publications.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The invention is further illustrated in the following non-limiting examples.

### EXAMPLES

### Example 1. Construction of Tg/TKO Mice

Mice carrying a heavy-chain antibody transgenic locus in germline configuration within a background that is silenced for endogenous heavy and light chain antibody expression (triple knock-out, or TKO) were created as previously described (WO2004/076618 and WO2003/000737, Ren et al. Genomics, 84, 686, 2004; Zou et al., J. Immunol., 170, 1354, 2003). Briefly, transgenic mice were derived following pronuclear microinjection of freshly fertilised oocytes with a yeast artificial chromosome (YAC) comprising a plethora of human V_{H}, D and J genes in combination with mouse immunoglobulin constant region genes lacking C_{H}1 domains, mouse enhancer and regulatory regions. Yeast artificial chromosomes (YACs) are vectors that can be employed for the cloning of very large DNA inserts in yeast. As well as comprising all three cis-acting structural elements essential for behaving like natural yeast chromosomes (an autonomously replicating sequence (ARS), a centromere (CEN) and two telomeres (TEL)), their capacity to accept large DNA inserts enables them to reach the minimum size (150 kb) required for chromosome-like stability and for fidelity of transmission in yeast cells. The construction and use of YACs is well known in the art (e.g., Bruschi, C. V. and Gjuracic, K. Yeast Artificial Chromosomes, ENCYCLOPEDIA OF LIFE SCIENCES 2002 Macmillan Publishers Ltd, Nature Publishing Group/www.els.net).

The YAC used was about 340 kb comprises 10 human heavy chain V genes in their natural configuration, human heavy chain D and J genes, a murine Cγ1 gene and a murine 3′ enhancer gene. It lacks the C_{H}1 exon. Specifically, the YAC comprised (from 5′ to 3′): telomere-yeast TRP1 marker gene-Centromere-23 human V genes-human D genes-human J genes-mouse μenhancer and switch-mouse Cγ1 (C_{H}1Δ) gene-mouse 3′ enhancer-Hygromycin resistant gene-yeast marker gene H/S3-telomere.

The transgenic founder mice were back-crossed with animals that lacked endogenous immunoglobulin expression to create the Tg/TKO lines used in the immunisation studies described.

### Example 2. Antigen for Immunisation

The immunisations used serum purified human and cyno serum albumin. Serum purified human (HSA) and cyno (CSA) serum albumin were purchased from Sigma (cat# A4327) and Abcam (cat# ab184894).

### Example 3. Immunisation Protocol

Three Crescendo mice aged 8-12 weeks of age each received an initial immunisation of 50 μg of CSA, emulsified in Complete Freund's Adjuvant and delivered subcutaneously, followed by 3 boosts of 10 μg of HSA, emulsified in Incomplete Freund's Adjuvant, also administered subcutaneously, given at weekly intervals following the initial priming. A final dose of HSA was administered intraperitoneally, in phosphate buffered saline, in the absence of adjuvant. At 49 days post the initial immunisation the mice were terminated, and brachial and inguinal lymph nodes and spleen were harvested into RNAlater (Qiagen cat#76104). Serum was collected and stored for testing for responses.

### Example 4. Serum ELISA

Nunc Maxisorp plates were coated overnight at 4° C. with HSA at 1 μg/ml in PBS solution. Plates were then washed using PBS supplemented with 0.05% Tween 20, followed by washes with PBS without added tween, and blocked with a solution of 3% skimmed milk powder (Marvel) in PBS for at least one hour at room temperature. Dilutions of serum in 3% Marvel/PBS were prepared in polypropylene tubes or plates and incubated for at least one hour at room temperature prior to transfer to the blocked ELISA plate where a further incubation of at least one hour took place. Following washing in PBS/Tween and PBS a solution of biotin-conjugated, goat anti mouse IgG, Fcgamma subclass 1 specific antibody (Jackson 115-065-205), prepared at 1:10000 dilution in PBS/3% Marvel was then added and plates were incubated at room temperature for at least one hour, then washed using PBS/Tween and PBS. Neutravidin-HRP solution (Pierce 31030) diluted 1:1000 in 3% Marvel/PBS was added to the ELISA plates and incubated for at least 30 minutes. Following further washing, the ELISA was developed using TMB substrate (Sigma cat. no. T0440) and the reaction was stopped after 10 minutes by the addition of 0.5M H2504 solution. Absorbances were determined by reading at 450 nm.

### Example 5. Generation of Libraries from Immunised Mice

a. Processing Tissues, RNA Extraction and cDNA Manufacture

Spleen, and lymph nodes were collected into RNAlater from each immunised animal. For each animal, ¼ of the spleen and 4 lymph nodes were processed separately. Initially, the tissues were homogenised; following with RNA precipitation. RNA purification was carried out on the QlAcube using the RNeasy 96 QlAcube kit and QlAcube HT plastics. Each RNA sample was then used to make cDNA using Superscript III RT-PCR high-fidelity kit.

b. Cloning into Phagemid Vector

A PCR-based method was used to clone the V_{H} cDNA libraries in the phagemid vector, pUCG3. Purified V_{H} RT-PCR products were employed as megaprimers with linearised vector pUCG3-C-tag to give phagemid products for phage library creation. The products of PCR were analysed on a 1% agarose gel. V_{H}/phagemid PCR products were pooled by animal-of-origin and purified using Thermo GeneJet PCR purification kit according to the manufacturer's instructions. Eluted DNA was used to transform TG1 E. coli (Lucigen, cat. no. 60502-2) by electroporation using the Bio-Rad GenePulser Xcell.

A 10-fold dilution series of the transformations was plated on 2xTY agar petri plates with 2% (w/v) glucose and 100 μg/ml ampicillin. Resulting colonies on these dishes were used to estimate library size. The remainder of the transformation was plated on large format 2xTY agar Bioassay dishes supplemented with 2% (w/v) glucose and 100 μg/ml ampicillin. All agar plates were incubated overnight at 30° C. Libraries were harvested by adding 10 ml of 2xTY broth to the large format bioassay dishes. Bacterial colonies were gently scraped and OD600 recorded. Aliquots were stored at −80oC in cryovials after addition of an equal volume of 50% (v/v) glycerol solution or used directly in a phage selection process

### Example 6. Selection Strategies for Isolation of Hsa-Binding V_{H}

To generate the Humabody® leads, the PEG precipitated phage library from the mice was used in panning selections with immuno-tube bound recombinant HSA protein at pH5 or pH7 to enrich for human serum albumin binding according to published methods (Antibody Engineering, Edited by Benny Lo, chapter 8, p161-176, 2004).

### Example 7. Assays for Target Binding

V_{H} from the different selections were screened in one or more of the following assays to identify V_{H} binding to HSA and CSA.

a. Beads Based FLISA Binding Assay for Human and Cyno Serum Albumin

For the lead Humabody V_{H} HSA194-D04 and HSA191E0-2 the periplasmic extract in plates was tested in homogenous high throughput assays using fluorescence microvolume assay technology in order to identify specific clones binding to the human and cynomolgus serum albumin. An assay was performed using Fluorescence Microvolume Assay technology which measures cell associated fluorescence within a defined volume at the bottom of the well of the assay plate in a homogenous assay format (Dietz et aL, Cytometry 23:177-186 (1996), Miraglia et al., J. Biomol. Screening 4:193-204 (1999). The assays were performed in 384 well assay format for analysis the data was subsequently reverted to the 96 well layout of the source periplasmic sample plates Small-scale bacterial periplasmic extracts were prepared from 1 ml cultures, grown in deep well plates. Starter cultures were used to inoculate 96-well deep well plates (Fisher, cat# MPA-600-030X) containing 2XTY broth (Melford, M2130), supplemented with 0.1% (w/v) glucose+100 ug/ml ampicillin at 30° C. with 250 rpm shaking. When OD600 had achieved 0.5-1, V_{H} production was induced by adding 100u1 of 2XTY, supplemented with IPTG (final concentration 5 mM) and ampicillin and the cultures were grown overnight at 30° C. with shaking at 220 rpm. E. coli were pelleted by centrifugation at 3200 rpm for 10 mins and supernatants discarded Cell pellets were resuspended in 120μI ice cold MES buffer (50 mM MOPS, 0.5 mM EDTA, 20% 0.5M Sucrose), then 180μI of 1:5 diluted ice-cold extraction buffer was added. Cells were incubated on ice for 30 minutes then centrifuged at 4500 rpm for 15 mins at 4° C. Supernatants were transferred to polypropylene plates and used, following incubation in 1× PBST blocking solution, directly in EL ISA.

Populations of beads: SOL-R4, SOL-R5 Tm carboxyl beads coupled to recombinant HSA Domain 1-11 (cat#9905), recombinant HSA Domain II (cat. #9902) and CSA. The beads were diluted to required concentrations.

b. Preparation of Purified V_{H}

V_{H} were purified from the supernatants of W3110 E coli with pJExpress vector. For this procedure up to 1 L cultures were grown at 37° C. with 250 rpm shaking in 2xTY broth (Melford, M2130), supplemented with 0.1% (w/v) glucose+50 ug/ml kanamycin with 250 rpm shaking. When OD600 had achieved 0.5-1, V_{H} production was induced by adding IPTG and kanamycin and the cultures were grown overnight at 30° C. with shaking at 250 rpm. E. coli were pelleted by centrifugation at 3200 rpm and the resulting supernatants were harvested and V_{H} purified using a Capture Select C-tag XL affinity matrix (Thermo Fisher Cat#2943072010), following with a Size exclusion Chromatography using a HiLoad 26/600 Superdrex 75 μg column on an AKTA Pure system.

Yields of purified V_{H} were estimated spectrophotometrically and purity was assessed using SDS PAGE.

c. Binding Kinetics.

Binding studies with human, cyno and mouse serum albumin in pH 7.4 and pH 6.0 were carried out using single cycle kinetics on a Biacore T200. Serum albumin (HSA, CSA, MSA) were coupled to CM5 chip by amine coupling using the amine reagent coupling kit GE Healthcare BR-1000-50. The surface of the chip was activated according to manufacturer's instructions. Serum albumin (HSA, CSA, MSA) were captured and cross-linked to the sensor chip surface by injecting of serum albumin (HSA, CSA, MSA) in 10 mM sodium acetate, pH 5.0. Following with the injection of 1M Ethanolamine to stabilise the serum albumin on the surface. With this procedure around 300 RU were immobilized.

For kinetics measurement four-fold serial dilution of the V_{H} single domain antibodies (194D04-2 or 191 E02-2) in either PBST pH 7.4 or PBST pH 6.0 were injected with 120s association and 300s dissociation at the flow rate of 45 μl/min at 25° C. Binding response was corrected by subtracting both the blank flow cell and from buffer run on the same flow cell. The traces were fitted using 1:1 model.

TABLE 2
Calculated kinetic constants and K_{D} for the Humabody V_{H} to serum albumin.

| | Human Serum Albumin (HSA) | | | Cyno Serum Albumin (CSA) | | | MSA |
|---|---|---|---|---|---|---|---|
| | K_{D} | ka (1/Ms) | kd (1/s) | K_{D} | ka (1/Ms) | kd (1/s) | K_{D} |
| SEQ ID | 5.9E−07 | 3.3E+05 | 1.9E−01 | 1.0E−07 | 2.9E+05 | 4.1E−02 | No binding |
| NO. 30 | | | | | | | |
| pH 7.4 | | | | | | | |
| SEQ ID | 6.0E−07 | 5.3E+05 | 3.2E−01 | 9.9E−08 | 6.4E+05 | 6.44E−02 | No binding |
| NO. 30 | | | | | | | |
| pH 6.0 | | | | | | | |
| SEQ ID | 3.0E−08 | 6.2E+05 | 1.9E−02 | 1.1E−08 | 5.7E+05 | 6.5E−03 | No binding |
| NO. 5 | | | | | | | |
| pH 7.4 | | | | | | | |
| SEQ ID | 3.7E−08 | 8.4E+05 | 3.1E−02 | 4.9E−09 | 6.4E+05 | 3.2E−03 | No binding |
| NO. 5 | | | | | | | |
| pH 6.0 | | | | | | | |

### Example 9 V_{H} Single Domain Antibodies Demonstrate Good Stability

Purified V_{H} were subjected to size exclusion chromatography. Briefly, purified V_{H} were stored at 10 mg/ml in selected buffer for 0-7 days at either 4° C. or 25° C., and then analysed at various time points using a Waters H-Class Bio UPLC containing a PDA detector (detection at 280 nm) with separation on a Waters ACQUITY BEH 125A SEC column. Samples were injected in 10 μl volumes and were run in a mobile phase containing 200 mM NaCl, 100 mM sodium phosphate, pH 7.4+5% propan-1-ol at a flow rate of 0.4 ml/min. Data were collected for 6 minutes and the percentage of monomeric protein in the sample after storage was calculated.

After incubation at 4° C. and 40° C. for 7 days, no significant change was seen.

TABLE 3
Stability of TPP-712 and 814. This shows the percentage of
monomer present after 0, 1, and 7 days.

| % Purity | % Purity by | | | | |
|---|---|---|---|---|---|
| by SEC | SEC 4oC | | % Purity by SEC 40oC | | |
| | 1 | 7 | 0 | 1 | 7 |
| TPP-712 | 99.48 | 99.35 | 99.48 | 99.15 | 96.66 |
| TPP-814 | 99.23 | 98.80 | 99.23 | 98.28 | 93.29 |

### Example 10 Pharmacokinetics Analysis of Single Intravenous Dose of Half-Life Extended Constructs in the Double Transgenic Humanised FcRn/Hsa Mouse

Briefly, male or female GenOway Human HSA/FcRn Tg mice were dosed with a single intravenous injection of compounds, listed in the table no 4 (n=3) at either 1 or 2 mg/kg via tail vein. Some of the constructs contained purification/detection tag such as: polyhistidine or FLAG tag. Blood samples were collected at pre-dose and at 0.083h, 1 h, 8h, 24 h, 48h, 72h and 96h post drug administration via the saphenous vein. At 168h post dose all animals were euthanised and blood was collected. Plasma was separated and stored at −80° C. until an assay was carried out. Plasma samples were analysed on the Gyrolab immunoassay platform, using as capture biotinylated human PSMA or human CD137 and either human CD137Dylight650, human PD-1 Dylight650 or anti-Flag-AF647 rabbit mAb (NEB, cat#15009S) as detection. Data was analysed using Gyros to obtain compound concentrations in plasma. Pharmacokinetic analysis of data was done using PK Solver 2.0, an Excel add on. Results of the study show that compounds have a half-life in the range of 19.9 to 78.7 hours when dosed at 1 or 2 mg/kg intravenously in human HSA/FcRn Tg mice.

TABLE 4
Summary table for pK parameters.

| | Format | Dose | Cmax | Half-life |
|---|---|---|---|---|
| Number | (VHs binding to specified target) | (mg/kg) | [ug/ml] | [h] |
| 814 protein sequence SEQ | PSMA-6GS-CD137-6GS-HSA The HSA | 1 | 17.5 | 40.7 |
| ID NO. 22, nucleic acid | binder is that shown in SEQ ID NO. 5 | | | |
| sequence SEQ ID NO. 23 | | | | |
| 712 protein sequence SEQ | PSMA-6GS-CD137-6GS-HSA The HSA | 1 | 23.8 | 22.5 |
| ID NO. 24, nucleic acid | binder is that shown in SEQ ID NO. 30 | | | |
| sequence SEQ ID NO. 25 | | | | |
| 446 protein sequence SEQ | PSMA-6GS-HSA The HSA binder is that | 2 | 43.9 | 26 |
| ID NO. 26, nucleic acid | shown in SEQ ID NO. 1 | | | |
| sequence, SEQ ID NO. 27 | | | | |
| 708 protein sequence SEQ | PSMA-6GS-HSA The HSA binder is that | 1 | 20.9 | 78.7 |
| ID NO. 28, nucleic acid | shown in SEQ ID NO. 5 | | | |
| sequence, SEQ ID NO. 29 | | | | |
| 1010 protein sequence SEQ | PD-1-6GS-HSA-6GS-CD137 The HSA | 2 | 53.7 | 20.3 |
| ID NO. 34, nucleic acid | binder is that shown in SEQ ID NO. 30 | | | |
| sequence SEQ ID NO. 37 | | | | |
| 1027 protein sequence SEQ | CD137-6GS-HSA-6GS-PD-1 The HSA | 2 | 54.5 | 19.9 |
| ID NO. 32, nucleic acid | binder is that shown in SEQ ID NO.30 | | | |
| sequence SEQ ID NO. 38 | | | | |
| 1028 protein sequence SEQ | CD137-6GS-HSA-6GS-PD-1 The HSA | 2 | 61.3 | 51.6 |
| ID NO. 33, nucleic acid | binder is that shown in SEQ ID NO. 5 | | | |
| sequence SEQ ID NO. 39 | | | | |

### Example 8 Pk Study in Cynomolgus Monkey

Prior to initiating the cynomolgus monkey PK study a review of replacement, reduction, and refinement considerations, as well as ethical and scientific justification (e.g. target expression/homology versus human, dose levels, etc), and risks, was conducted both at Crescendo Biologics and the contract research organisation. The animal work in this cynomolgus monkey PK study was conducted under a UK Home Office Project License at a contract research organisation based in the UK. The Home Office license governing this study strictly specifies the limits of severity of effects on the animals. The procedures in the protocol did not cause any effects which exceeded the severity limit of the procedure.

Briefly, three male cynomolgus macaque were dosed with 4 mg/kg of Humabody construct listed in the table (TPP-1246), the study was carried out in Charles River Study. Serum samples were taken at Predose (˜24 hrs), 1 hr, 2 hrs, 4 hrs, 8 hrs, 24 hrs, 48 hrs, 72 hrs, 120 hrs, 168 hrs, 216 hrs, 264 hrs, 312 hrs, 360 hrs, 408 hrs and 504 hrs post dose from all test subjects and frozen prior to testing. PK analysis was performed on the serum samples using assays developed at Crescendo.

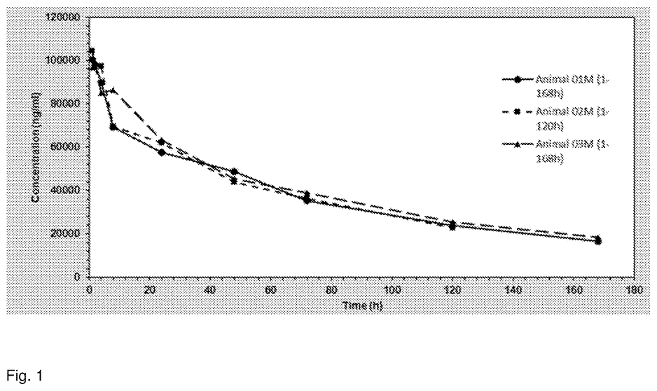

The PK assay utilises the Gyrolab Xplore immunoassay platform using a sandwich immunoassay format; the analyte (listed in the table) is immobilized by biotinylated CD137 antigen and is detected by dyLight650 labelled PD-1. The assay was optimised and established to confirm range and reproducibility and the sample analysis was completed in accordance with the established assays. The PK analysis was performed on the PK data from the following timepoints: Animal 01: 1 to 168 hrs, Animal 02: 1 to 120 hrs and Animal 03: 1 to 168 hrs. The reported PK parameters are the mean from the 3 individuals for each result. The T½ of TPP-1246 in Cyno Serum has been demonstrated to be 84.5 hrs ±7.58 hrs. Data is shown in FIG. 1.

TABLE 5
PK parameter estimates following a single intravenous dose of TPP-1246 in cynomolgus macaque

| | | Dose | Cmax | Half-life |
|---|---|---|---|---|
| TPP | Compound name | (mg/kg) | [ug/ml] | [h] |
| 1246 protein sequence | CD137-6GS-HSA-6GS-PD-1 | 4 | 100.6939 | 84.51278 |
| SEQ ID NO. 35, | The HSA binder is that | | | |
| nucleic acid sequence | shown in SEQ ID NO.30 | | | |
| SEQ ID NO. 36 | | | | |

Molecules used in all experiments above were based on the molecules as stated above. Tested molecules included, where appropriate, C-terminal sequences, such as purification tags.

1. An immunoglobulin single variable domain antibody that binds to human HSA comprising
a) a CDR1 having SEQ ID NO. 2 or an amino acid sequence that has 1 or 2 differences with SEQ ID NO. 2;
b) a CDR2 having SEQ ID NO. 3 or an amino acid sequence that has 1, 2, 3, 4, 5, 6 differences with SEQ ID NO. 3 and
c) a CDR3 having SEQ ID NO. 4 or an amino acid sequence that has 1, 2, 3 or 4 differences with SEQ ID NO. 4.

2. The immunoglobulin single variable domain antibody according to claim 1 comprising or consisting of SEQ ID NO. 1 or a sequence with at least 80%, 90% or 95% homology thereto.

3. The immunoglobulin single variable domain antibody according to claim 2 comprising or consisting of SEQ ID NO. 30 or a sequence with at least 80%, 90% or 95% homology thereto.

4. An immunoglobulin single variable domain antibody that binds to human HSA comprising
d) a CDR1 having SEQ ID NO. 6 or an amino acid sequence that has 1 or 2 differences with SEQ ID NO. 6;
e) a CDR2 having SEQ ID NO. 7 or an amino acid sequence that has 1, 2, 3, 4, 5, 6 differences with SEQ ID NO. 7 and
f) a CDR3 having SEQ ID NO. 8 or an amino acid sequence that has 1, 2, 3 or 4 differences with SEQ ID NO. 8.

5. The immunoglobulin single variable domain antibody according to claim 4 comprising or consisting of SEQ ID NO. 5 or a sequence with at least 80%, 90% or 95% homology thereto.

6. A protein or construct comprising an immunoglobulin single variable domain according to a preceding claim.

7. The protein or construct according to claim 6 comprising a therapeutic moiety.

8. The protein or construct according to claim 7 wherein said therapeutic moiety is an antibody or fragment thereof.

9. The protein or construct according to claim 8 wherein said fragment is an scFv, Fv, heavy chain or single domain antibody.

10. The protein or construct according to claim 9 wherein said single domain antibody is a single variable heavy chain domain antibody.

11. The protein or construct according to any of claims 6 to 10 wherein the immunoglobulin single variable domain antibody is linked to the therapeutic moiety with a peptide linker.

12. The protein or construct according to claim 10 wherein said peptide linker is (G4S)n wherein n is 1 to 15.

13. The protein or construct according to any of claims 6 to 12 wherein the immunoglobulin single variable domain antibody is located at the N or C terminal end of the protein.

14. The protein or construct according to claim 13 wherein the immunoglobulin single variable domain antibody is located at the C terminal end of the protein and comprises a C terminal extension of 1 to 50 amino acids.

15. A method for extending the half life of a protein comprising joining said protein to an immunoglobulin single variable domain antibody according to any of claims 1 to 5.

16. The use of an immunoglobulin single variable domain antibody according to any of claims 1 to 5 in extending the half life of a therapeutic moiety when said an immunoglobulin single variable domain according to any of claims is linked to said therapeutic moiety in a fusion protein.

17. A pharmaceutical composition comprising an immunoglobulin single variable domain according to any of claims 1 to 5 or a protein or construct according to any of claims 6 to 14.

18. A nucleic acid sequence that encodes an amino acid sequence according to any of claims 1 to 5.

19. The nucleic acid sequence according to claim 18 comprising SEQ ID NO. 16.

20. The nucleic acid sequence of claim 18 or 19 wherein said nucleic acid sequence is linked with a linker to a second nucleic acid sequence.

21. The nucleic acid sequence of claim 20 wherein said second nucleic acid encodes a therapeutic moiety.

22. The nucleic acid sequence of claim 20 or 21 wherein said linker is a nucleic acid linker.

23. A vector comprising a nucleic acid sequence according to any of claims 18 to 22.

24. A host cell comprising the nucleic acid sequence according to any of claims 18 to 22 or a vector of claim 23.

25. A kit comprising an immunoglobulin single variable domain antibody according to any of claims 1 to 5 or a protein or construct according to any of claims 6 to 14 or a pharmaceutical composition according to claim 16.

26. A method for producing a binding molecule comprising at least one human immunoglobulin single domain antibody according to any of claims 6 to 14 capable of binding human HSA wherein said domain is a human V_{H} domain said method comprising
a) immunising a transgenic mouse with an HSA antigen wherein said mouse expresses a nucleic acid construct comprising human heavy chain V genes and is not capable of making functional endogenous light or heavy chains, b) generating a library of sequences comprising V_{H} domain sequences from said mouse and c) isolating sequences comprising V_{H} domain sequences from said libraries.


SEQUENCE LISTING (39 sequences)

SEQ ID NO: 1 - protein, 122 aa, Homo sapiens
EVQLLESGGGLVKPGGSLRLSCAASGFTFSNYNMNWVRQAPGKRLEWVSSISSAGTHIYSADSVKGRFTISRDNAKNSLYLQMNSLRAEDTGVYYCARDPHSTGWYKDFDYWGQGTLVTVSS

SEQ ID NO: 2 - protein, 5 aa, Homo sapiens
NYNMN

SEQ ID NO: 3 - protein, 17 aa, Homo sapiens
SISSAGTHIYSADSVKG

SEQ ID NO: 4 - protein, 13 aa, Homo sapiens
DPHSTGWYKDFDY

SEQ ID NO: 5 - protein, 122 aa, Homo sapiens
EVQLLESGGGLVKPGGSLRLSCAASGFTVSSYTMNWVRQAPGKGLEWVSSISSSGRYIYYADSVKGRFTISRDNAKNSLYLQMNSLRAEDTAVYYCARDPRMVGNPHEFDIWGQGTMVTVSS

SEQ ID NO: 6 - protein, 5 aa, Homo sapiens
SYTMN

SEQ ID NO: 7 - protein, 17 aa, Homo sapiens
SISSSGRYIYYADSVKG

SEQ ID NO: 8 - protein, 13 aa, Homo sapiens
DPRMVGNPHEFDI

SEQ ID NO: 9 - protein, 609 aa, Homo sapiens [sequence omitted]

SEQ ID NO: 10 - protein, 5 aa, Artificial Sequence
  C-terminal extension
VTVSS

SEQ ID NO: 11 - protein, 6 aa, Artificial Sequence
  His-tag
HHHHHH

SEQ ID NO: 12 - protein, 5 aa, Artificial Sequence
  Gly Ser linker
GGGGS

SEQ ID NO: 13 - protein, 10 aa, Artificial Sequence
  Gly Ser linker
GGGGSGGGGS

SEQ ID NO: 14 - protein, 10 aa, Artificial Sequence
  Gly Ser linker
SGGGGSGGGG

SEQ ID NO: 15 - protein, 14 aa, Artificial Sequence
  Gly Ser linker
GGGGSGGGGSGGGG

SEQ ID NO: 16 - protein, 8 aa, Artificial Sequence
  Gly Ser linker
GSGSGSGS

SEQ ID NO: 17 - protein, 8 aa, Artificial Sequence
  Gly Ser linker
GGSGSGSG

SEQ ID NO: 18 - protein, 6 aa, Artificial Sequence
  Gly Ser linker
GGSGSG

SEQ ID NO: 19 - protein, 4 aa, Artificial Sequence
  Gly Ser linker
GGSG

SEQ ID NO: 20 - DNA, 366 bp, Homo sapiens
gaggtgcagctgttggagtctgggggaggcctggtcaagcctggggggtccctgagactctcctgtgcagcctctggattcaccttcagtaactataacatgaactgggtccgccaggctccagggaagaggctggagtgggtctcatcgattagtagtgctggtactcacatatactccgcagactcagtgaagggccgattcaccatctccagagacaacgccaagaactcactgtatctgcaaatgaacagcctgagagccgaggacacaggtgtttattactgtgcgagagatcctcatagcactggctggtacaaggactttgactactggggccagggaaccctggtcaccgtctcctca

SEQ ID NO: 21 - DNA, 366 bp, Homo sapiens
gaggtgcagctgttggagtctgggggaggcctggtcaagccgggggggtccctgagactctcctgtgcagcctctggattcaccgtcagtagctataccatgaactgggtccgccaggctccggggaaggggctggagtgggtctcatccattagtagtagtggtcgttacatatactacgcagactcagtgaagggccgattcaccatctccagagacaacgccaagaactcattatatctgcaaatgaacagcctgagagccgaggacacagctgtatattattgtgcgagagatccccgtatggttgggaacccccatgaatttgatatctggggccaagggacaatggtcaccgtctcctca

SEQ ID NO: 22 - protein, 422 aa, Artificial Sequence
  TPP-814 construct
EVQLVESGGGVVQPGRSLRLSCAASGFSFSGYGMHWVRQAPGKGLEWVAYISYDGSNKYYADSVKGRFTISRDNSKNTLYLQMNSLRAEDTAVYYCAKDPAWGLRLGESSSYDFDIWGQGTMVTVSSGGGGSGGGGSGGGGSGGGGSGGGGSGGGGSEVQLVESGGGLVQPGGSLRLSCAASGFTLSNYWMNWVRQAPGKGLEWVANINQDGSERYYVDSVKGRFTISRDNAKNSLYLQMNSLRAEDTAVYYCARGGEGYGVDHYGLDVSGQGTTVTVSSGGGGSGGGGSGGGGSGGGGSEVQLLESGGGLVKPGGSLRLSCAASGFTVSSYTMNWVRQAPGKGLEWVSSISSSGRYIYYADSVKGRFTISRDNAKNSLYLQMNSLRAEDTAVYYCARDPRMVGNPHEFDIWGQGTMVTVSS

SEQ ID NO: 23 - DNA, 1266 bp, Artificial Sequence
  TPP-814 construct
gaggtgcagctggtggagtctgggggaggcgtggtccagcctgggaggtccctgagactctcctgtgcagcctctggattctccttcagtggctatggcatgcactgggtccgccaggctccaggcaagggactggagtgggtggcatatatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagatccggcctggggattacgtttgggggagtcatcgtcctatgattttgatatctggggccaagggacaatggtcaccgtctcctcaggtggtggcggttcaggcggaggtggctctggaggtggaggttcaggaggtggtggttctggcggcggtggatcgggtggaggtggtagtgaggtgcagttagttgagagcggaggtggtttagttcagccggggggctcgcttcgcctgtcgtgcgccgcctcgggattcacattatcaaactactggatgaattgggtccgccaggctccgggcaaaggtcttgagtgggtggcgaacattaatcaggacgggagcgagcgttattacgttgattcggtaaaaggacgtttcactatcagtcgtgacaacgctaaaaattccttgtacttacagatgaactcacttcgtgctgaggacaccgcagtgtactactgtgctcgcggtggtgaaggatacggcgtcgatcactacggccttgatgtatcaggacaggggactacagttaccgtctcttccggaggtggaggttcaggaggtggtggttctggtggtggcggttcaggtggaggtggtagtgaggtgcagctgttggagtctgggggaggcctggtcaagccgggggggtccctgagactctcctgtgcagcctctggattcaccgtcagtagctataccatgaactgggtccgccaggctccggggaaggggctggagtgggtctcatccattagtagtagtggtcgttacatatactacgcagactcagtgaagggccgattcaccatctccagagacaacgccaagaactcattatatctgcaaatgaacagcctgagagccgaggacacagctgtatattattgtgcgagagatccccgtatggttgggaacccccatgaatttgatatctggggccaagggacaatggtcaccgtctcctca

SEQ ID NO: 24 - protein, 432 aa, Artificial Sequence
  TPP-712 construct
EVQLVESGGGVVQPGRSLRLSCAASGFSFSGYGMHWVRQAPGKGLEWVAYISYDGSNKYYADSVKGRFTISRDNSKNTLYLQMNSLRAEDTAVYYCAKDPAWGLRLGESSSYDFDIWGQGTMVTVSSGGGGSGGGGSGGGGSGGGGSGGGGSGGGGSEVQLVESGGGLVQPGGSLRLSCAASGFTLSNYWMNWVRQAPGKGLEWVANINQDGSERYYVDSVKGRFTISRDNAKNSLYLQMNSLRAEDTAVYYCARGGEGYGVDHYGLDVSGQGTTVTVSSGGGGSGGGGSGGGGSGGGGSGGGGSGGGGSEVQLVESGGGLVKPGGSLRLSCAASGFTFSNYNMNWVRQAPGKGLEWVSSISSAGTHIYYADSVKGRFTISRDNAKNSLYLQMNSLRAEDTAVYYCARDPHSTGWYKDFDYWGQGTLVTVSS

SEQ ID NO: 25 - DNA, 1296 bp, Artificial Sequence
  TPP-712 construct
gaggtgcagctggtggagtctgggggaggcgtggtccagcctgggaggtccctgagactctcctgtgcagcctctggattctccttcagtggctatggcatgcactgggtccgccaggctccaggcaagggactggagtgggtggcatatatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagatccggcctggggattacgtttgggggagtcatcgtcctatgattttgatatctggggccaagggacaatggtcaccgtctcctcaggtggtggcggttcaggcggaggtggctctggaggtggaggttcaggaggtggtggttctggcggcggtggatcgggtggaggtggtagtgaggtgcagttagttgagagcggaggtggtttagttcagccggggggctcgcttcgcctgtcgtgcgccgcctcgggattcacattatcaaactactggatgaattgggtccgccaggctccgggcaaaggtcttgagtgggtggcgaacattaatcaggacgggagcgagcgttattacgttgattcggtaaaaggacgtttcactatcagtcgtgacaacgctaaaaattccttgtacttacagatgaactcacttcgtgctgaggacaccgcagtgtactactgtgctcgcggtggtgaaggatacggcgtcgatcactacggccttgatgtatcaggacaggggactacagttaccgtctcttccggtggtggcggttcaggcggaggtggctctggaggtggaggttcaggaggtggtggttctggcggcggtggatcgggtggaggtggtagtgaggtgcagctggtggagtctgggggaggcctggtcaagcctggggggtccctgagactctcctgtgcagcctctggattcaccttcagtaactataacatgaactgggtccgccaggctccagggaaggggctggagtgggtctcatcgattagtagtgctggtactcacatatactacgcagactcagtgaagggccgattcaccatctccagagacaacgccaagaactcactgtatctgcaaatgaacagcctgagagccgaggacacagctgtttattactgtgcgagagatcctcatagcactggctggtacaaggactttgactactggggccagggaaccctggtcaccgtctcctca

SEQ ID NO: 26 - protein, 265 aa, Artificial Sequence
  TPP-446 construct
EVQLLESGGGLVQPGGSLRLSCAASGFSFSSYALSWVRQAPGKGLEWVSSIGENDGTTDYADAVKGRFTISRDNSKNTLYLQMNSLRVEDTAVYYCVKDGVHWGQGTLVTVSSGGGGSGGGGSGGGGSGGGGSGGGGSGGGGSEVQLLESGGGLVKPGGSLRLSCAASGFTFSNYNMNWVRQAPGKRLEWVSSISSAGTHIYSADSVKGRFTISRDNAKNSLYLQMNSLRAEDTGVYYCARDPHSTGWYKDFDYWGQGTLVTVSS

SEQ ID NO: 27 - DNA, 795 bp, Artificial Sequence
  TPP-446 construct
gaggtgcagctgttggagtctgggggaggcttggtacagcctggggggtccctgagactctcctgtgcagcctctggattcagttttagcagctatgccctcagttgggtccgccaggctccagggaaggggctggagtgggtctcaagtattggtgagaatgatggtaccacagactacgcagacgccgtgaagggccgattcaccatctccagagacaattccaagaatacgctgtatctgcaaatgaacagcctgagagtcgaggacacggccgtctattactgtgtgaaagatggtgtccactggggccagggaaccctggtcaccgtctcctcaggtggtggcggttcaggcggaggtggctctggaggtggaggttcaggaggtggtggttctggcggcggtggatcgggtggaggtggtagtgaggtgcagctgttggagtctgggggaggcctggtcaagcctggggggtccctgagactctcctgtgcagcctctggattcaccttcagtaactataacatgaactgggtccgccaggctccagggaagaggctggagtgggtctcatcgattagtagtgctggtactcacatatactccgcagactcagtgaagggccgattcaccatctccagagacaacgccaagaactcactgtatctgcaaatgaacagcctgagagccgaggacacaggtgtttattactgtgcgagagatcctcatagcactggctggtacaaggactttgactactggggccagggaaccctggtcaccgtctcctca

SEQ ID NO: 28 - protein, 265 aa, Artificial Sequence
  TPP-708 construct
EVQLLESGGGLVQPGGSLRLSCAASGFSFSSYALSWVRQAPGKGLEWVSSIGENDGTTDYADAVKGRFTISRDNSKNTLYLQMNSLRVEDTAVYYCVKDGVHWGQGTLVTVSSGGGGSGGGGSGGGGSGGGGSGGGGSGGGGSEVQLLESGGGLVKPGGSLRLSCAASGFTVSSYTMNWVRQAPGKGLEWVSSISSSGRYIYYADSVKGRFTISRDNAKNSLYLQMNSLRAEDTAVYYCARDPRMVGNPHEFDIWGQGTMVTVSS

SEQ ID NO: 29 - DNA, 795 bp, Artificial Sequence
  TPP-708 construct
gaggtgcagctgttggagtctgggggaggcttggtacagcctggggggtccctgagactctcctgtgcagcctctggattcagttttagcagctatgccctcagttgggtccgccaggctccagggaaggggctggagtgggtctcaagtattggtgagaatgatggtaccacagactacgcagacgccgtgaagggccgattcaccatctccagagacaattccaagaatacgctgtatctgcaaatgaacagcctgagagtcgaggacacggccgtctattactgtgtgaaagatggtgtccactggggccagggaaccctggtcaccgtctcctcaggtggtggcggttcaggcggaggtggctctggaggtggaggttcaggaggtggtggttctggcggcggtggatcgggtggaggtggtagtgaggtgcagctgttggagtctgggggaggcctggtcaagccgggggggtccctgagactctcctgtgcagcctctggattcaccgtcagtagctataccatgaactgggtccgccaggctccggggaaggggctggagtgggtctcatccattagtagtagtggtcgttacatatactacgcagactcagtgaagggccgattcaccatctccagagacaacgccaagaactcattatatctgcaaatgaacagcctgagagccgaggacacagctgtatattattgtgcgagagatccccgtatggttgggaacccccatgaatttgatatctggggccaagggacaatggtcaccgtctcctca

SEQ ID NO: 30 - protein, 122 aa, Homo sapiens
EVQLVESGGGLVKPGGSLRLSCAASGFTFSNYNMNWVRQAPGKGLEWVSSISSAGTHIYYADSVKGRFTISRDNAKNSLYLQMNSLRAEDTAVYYCARDPHSTGWYKDFDYWGQGTLVTVSS

SEQ ID NO: 31 - DNA, 366 bp, Homo sapiens
gaggtgcagctggtggagtctgggggaggcctggtcaagcctggggggtccctgagactctcctgtgcagcctctggattcaccttcagtaactataacatgaactgggtccgccaggctccagggaaggggctggagtgggtctcatcgattagtagtgctggtactcacatatactacgcagactcagtgaagggccgattcaccatctccagagacaacgccaagaactcactgtatctgcaaatgaacagcctgagagccgaggacacagctgtttattactgtgcgagagatcctcatagcactggctggtacaaggactttgactactggggccagggaaccctggtcaccgtctcctca

SEQ ID NO: 32 - protein, 437 aa, Artificial Sequence
  TPP-1027 construct
EVQLVESGGGLVQPGGSLRLSCAASGFTLSNYWMNWVRQAPGKGLEWVANINQDGSERYYVDSVKGRFTISRDNAKNSLYLQMNSLRAEDTAVYYCARGGEGYGVDHYGLDVSGQGTTVTVSSGGGGSGGGGSGGGGSGGGGSGGGGSGGGGSEVQLVESGGGLVKPGGSLRLSCAASGFTFSNYNMNWVRQAPGKGLEWVSSISSAGTHIYYADSVKGRFTISRDNAKNSLYLQMNSLRAEDTAVYYCARDPHSTGWYKDFDYWGQGTLVTVSSGGGGSGGGGSGGGGSGGGGSGGGGSGGGGSEVQLVESGGGVVRPGGSLRLSCAASGFTFDDYAMSWVRQAPGKGLEWVSGITWNGGSTGYADSVKDRFTISRDNAKNSLYLQMNSLRAEDTALYYCVRDKYSYAWSYDDFDIWGQGTMVTVSSAAAHHHHHH

SEQ ID NO: 33 - protein, 437 aa, Artificial Sequence
  TPP-1028 construct
EVQLVESGGGLVQPGGSLRLSCAASGFTLSNYWMNWVRQAPGKGLEWVANINQDGSERYYVDSVKGRFTISRDNAKNSLYLQMNSLRAEDTAVYYCARGGEGYGVDHYGLDVSGQGTTVTVSSGGGGSGGGGSGGGGSGGGGSGGGGSGGGGSEVQLLESGGGLVKPGGSLRLSCAASGFTVSSYTMNWVRQAPGKGLEWVSSISSSGRYIYYADSVKGRFTISRDNAKNSLYLQMNSLRAEDTAVYYCARDPRMVGNPHEFDIWGQGTMVTVSSGGGGSGGGGSGGGGSGGGGSGGGGSGGGGSEVQLVESGGGVVRPGGSLRLSCAASGFTFDDYAMSWVRQAPGKGLEWVSGITWNGGSTGYADSVKDRFTISRDNAKNSLYLQMNSLRAEDTALYYCVRDKYSYAWSYDDFDIWGQGTMVTVSSAAAHHHHHH

SEQ ID NO: 34 - protein, 437 aa, Artificial Sequence
  TPP1010 - construct
EVQLVESGGGVVRPGGSLRLSCAASGFTFDDYAMSWVRQAPGKGLEWVSGITWNGGSTGYADSVKDRFTISRDNAKNSLYLQMNSLRAEDTALYYCVRDKYSYAWSYDDFDIWGQGTMVTVSSGGGGSGGGGSGGGGSGGGGSGGGGSGGGGSEVQLVESGGGLVKPGGSLRLSCAASGFTFSNYNMNWVRQAPGKGLEWVSSISSAGTHIYYADSVKGRFTISRDNAKNSLYLQMNSLRAEDTAVYYCARDPHSTGWYKDFDYWGQGTLVTVSSGGGGSGGGGSGGGGSGGGGSGGGGSGGGGSEVQLVESGGGVVQPGGSLRLSCAASGFTFSNYGMHWVRQAPGKGLEWVAVISNDGNTKTYADSVKGRFTISRDNSKNTLYLQMNSLRAEDTAVYYCAQDETTTVTASYYFDYWGQGTLVTVSSAAAHHHHHH

SEQ ID NO: 35 - protein, 428 aa, Artificial Sequence
  TPP-1246 construct
EVQLVESGGGLVQPGGSLRLSCAASGFTLSNYWMNWVRQAPGKGLEWVANINQDGSERYYVDSVKGRFTISRDNAKNSLYLQMNSLRAEDTAVYYCARGGEGYGVDHYGLDVSGQGTTVTVSSGGGGSGGGGSGGGGSGGGGSGGGGSGGGGSEVQLVESGGGLVKPGGSLRLSCAASGFTFSNYNMNWVRQAPGKGLEWVSSISSAGTHIYYADSVKGRFTISRDNAKNSLYLQMNSLRAEDTAVYYCARDPHSTGWYKDFDYWGQGTLVTVSSGGGGSGGGGSGGGGSGGGGSGGGGSGGGGSEVQLVESGGGVVRPGGSLRLSCAASGFTFDDYAMSWVRQAPGKGLEWVSGITWNGGSTGYADSVKDRFTISRDNAKNSLYLQMNSLRAEDTALYYCVRDKYSYAWSYDDFDIWGQGTMVTVSS

SEQ ID NO: 36 - DNA, 1284 bp, Artificial Sequence
  TPP-1246 construct
gaggtgcagttagttgagagcggaggtggtttagttcagccggggggctcgcttcgcctgtcgtgcgccgcctcgggattcacattatcaaactactggatgaattgggtccgccaggctccgggcaaaggtcttgagtgggtggcgaacattaatcaggacgggagcgagcgttattacgttgattcggtaaaaggacgtttcactatcagtcgtgacaacgctaaaaattccttgtacttacagatgaactcacttcgtgctgaggacaccgcagtgtactactgtgctcgcggtggtgaaggatacggcgtcgatcactacggccttgatgtatcaggacaggggactacagttaccgtctcttccggtggtggcggttcaggcggaggtggctctggaggtggaggttcaggaggtggtggttctggcggcggtggatcgggtggaggtggtagtgaggtgcagctggtggagtctgggggaggcctggtcaagcctggggggtccctgagactctcctgtgcagcctctggattcaccttcagtaactataacatgaactgggtccgccaggctccagggaaggggctggagtgggtctcatcgattagtagtgctggtactcacatatactacgcagactcagtgaagggccgattcaccatctccagagacaacgccaagaactcactgtatctgcaaatgaacagcctgagagccgaggacacagctgtttattactgtgcgagagatcctcatagcactggctggtacaaggactttgactactggggccagggaaccctggtcaccgtctcctcaggtggtggcggttcaggcggaggtggctctggaggtggaggttcaggaggtggtggttctggcggcggtggatcgggtggaggtggtagtgaggtgcagctggtggagtctgggggaggtgtggtacggcctggggggtccctgagactctcctgtgcagcctctggattcacctttgatgattatgccatgagttgggtccgccaagctccagggaaggggctggagtgggtctctggtattacttggaatggtggtagcacaggttatgcagactctgtgaaggaccgattcaccatctccagagacaacgccaagaactccctgtatctgcaaatgaacagtctgagagccgaggacacggccttgtattactgtgtgagagacaagtatagctatgcctggtcttatgatgattttgatatctggggccaagggacaatggtcaccgtctcctca

SEQ ID NO: 37 - DNA, 1311 bp, Artificial Sequence
  TPP-1010 construct
gaggtgcagctggtggagtctgggggaggtgtggtacggcctggggggtccctgagactctcctgtgcagcctctggattcacctttgatgattatgccatgagttgggtccgccaagctccagggaaggggctggagtgggtctctggtattacttggaatggtggtagcacaggttatgcagactctgtgaaggaccgattcaccatctccagagacaacgccaagaactccctgtatctgcaaatgaacagtctgagagccgaggacacggccttgtattactgtgtgagagacaagtatagctatgcctggtcttatgatgattttgatatctggggccaagggacaatggtcaccgtctcctcaggtggtggcggttcaggcggaggtggctctggaggtggaggttcaggaggtggtggttctggcggcggtggatcgggtggaggtggtagtgaggtgcagctggtggagtctgggggaggcctggtcaagcctggggggtccctgagactctcctgtgcagcctctggattcaccttcagtaactataacatgaactgggtccgccaggctccagggaaggggctggagtgggtctcatcgattagtagtgctggtactcacatatactacgcagactcagtgaagggccgattcaccatctccagagacaacgccaagaactcactgtatctgcaaatgaacagcctgagagccgaggacacagctgtttattactgtgcgagagatcctcatagcactggctggtacaaggactttgactactggggccagggaaccctggtcaccgtctcctcaggcggaggtggctctggaggaggaggttcaggaggtggtggatctggaggaggcggatcggggggtggaggaagtggcggcggtggtagtgaggtgcagctggtggagtctgggggaggcgttgtccagcctggggggtccctgagactctcctgtgcagcctctggattcaccttcagtaattatggcatgcactgggtccgccaggctccaggcaagggcctggagtgggtggcagtcatatcaaatgatggaaatactaaaacctatgcagattccgtgaagggccgattcaccatctccagagacaattccaagaacacgttatatctgcaaatgaacagcctgagagctgaggacacggctgtgtattactgtgcgcaagatgagacgactacagtaactgcgtcgtattactttgactactggggccagggaaccctggtcaccgtctcctcagcggccgcacaccaccatcatcaccac

SEQ ID NO: 38 - DNA, 1311 bp, Artificial Sequence
  TPP-1027 construct
gaggtgcagttagttgagagcggaggtggtttagttcagccggggggctcgcttcgcctgtcgtgcgccgcctcgggattcacattatcaaactactggatgaattgggtccgccaggctccgggcaaaggtcttgagtgggtggcgaacattaatcaggacgggagcgagcgttattacgttgattcggtaaaaggacgtttcactatcagtcgtgacaacgctaaaaattccttgtacttacagatgaactcacttcgtgctgaggacaccgcagtgtactactgtgctcgcggtggtgaaggatacggcgtcgatcactacggccttgatgtatcaggacaggggactacagttaccgtctcttccggtggtggcggttcaggcggaggtggctctggaggtggaggttcaggaggtggtggttctggcggcggtggatcgggtggaggtggtagtgaggtgcagctggtggagtctgggggaggcctggtcaagcctggggggtccctgagactctcctgtgcagcctctggattcaccttcagtaactataacatgaactgggtccgccaggctccagggaaggggctggagtgggtctcatcgattagtagtgctggtactcacatatactacgcagactcagtgaagggccgattcaccatctccagagacaacgccaagaactcactgtatctgcaaatgaacagcctgagagccgaggacacagctgtttattactgtgcgagagatcctcatagcactggctggtacaaggactttgactactggggccagggaaccctggtcaccgtctcctcaggtggtggcggttcaggcggaggtggctctggaggtggaggttcaggaggtggtggttctggcggcggtggatcgggtggaggtggtagtgaggtgcagctggtggagtctgggggaggtgtggtacggcctggggggtccctgagactctcctgtgcagcctctggattcacctttgatgattatgccatgagttgggtccgccaagctccagggaaggggctggagtgggtctctggtattacttggaatggtggtagcacaggttatgcagactctgtgaaggaccgattcaccatctccagagacaacgccaagaactccctgtatctgcaaatgaacagtctgagagccgaggacacggccttgtattactgtgtgagagacaagtatagctatgcctggtcttatgatgattttgatatctggggccaagggacaatggtcaccgtctcctcagcggccgcacaccaccatcatcaccac

SEQ ID NO: 39 - DNA, 1311 bp, Artificial Sequence
  TPP-1028 construct
gaggtgcagttagttgagagcggaggtggtttagttcagccggggggctcgcttcgcctgtcgtgcgccgcctcgggattcacattatcaaactactggatgaattgggtccgccaggctccgggcaaaggtcttgagtgggtggcgaacattaatcaggacgggagcgagcgttattacgttgattcggtaaaaggacgtttcactatcagtcgtgacaacgctaaaaattccttgtacttacagatgaactcacttcgtgctgaggacaccgcagtgtactactgtgctcgcggtggtgaaggatacggcgtcgatcactacggccttgatgtatcaggacaggggactacagttaccgtctcttccggtggtggcggttcaggcggaggtggctctggaggtggaggttcaggaggtggtggttctggcggcggtggatcgggtggaggtggtagtgaggtgcagctgttggagtctgggggaggcctggtcaagccgggggggtccctgagactctcctgtgcagcctctggattcaccgtcagtagctataccatgaactgggtccgccaggctccggggaaggggctggagtgggtctcatccattagtagtagtggtcgttacatatactacgcagactcagtgaagggccgattcaccatctccagagacaacgccaagaactcattatatctgcaaatgaacagcctgagagccgaggacacagctgtatattattgtgcgagagatccccgtatggttgggaacccccatgaatttgatatctggggccaagggacaatggtcaccgtctcctcaggtggtggcggttcaggcggaggtggctctggaggtggaggttcaggaggtggtggttctggcggcggtggatcgggtggaggtggtagtgaggtgcagctggtggagtctgggggaggtgtggtacggcctggggggtccctgagactctcctgtgcagcctctggattcacctttgatgattatgccatgagttgggtccgccaagctccagggaaggggctggagtgggtctctggtattacttggaatggtggtagcacaggttatgcagactctgtgaaggaccgattcaccatctccagagacaacgccaagaactccctgtatctgcaaatgaacagtctgagagccgaggacacggccttgtattactgtgtgagagacaagtatagctatgcctggtcttatgatgattttgatatctggggccaagggacaatggtcaccgtctcctcagcggccgcacaccaccatcatcaccac